SEQUENCE LISTING (28 sequences)

SEQ ID NO: 1 - DNA, 8 bp, Homo sapiens
taactggg

SEQ ID NO: 2 - DNA, 7 bp, Homo sapiens
cacagtg

SEQ ID NO: 3 - DNA, 9 bp, Homo sapiens
acaaaaacc

SEQ ID NO: 4 - DNA, 22 bp, Homo sapiens
ggagaccaccaaaccctccaaa

SEQ ID NO: 5 - DNA, 22 bp, Homo sapiens
gagagttgcagaaggggtgact

SEQ ID NO: 6 - DNA, 25 bp, Homo sapiens
ggaattcaagaacttggaccactgt

SEQ ID NO: 7 - DNA, 29 bp, Homo sapiens
cccccaagcttctttccaggctcctgggc

SEQ ID NO: 8 - DNA, 22 bp, Homo sapiens
gagagacacaggcacgtagaag

SEQ ID NO: 9 - DNA, 20 bp, Homo sapiens
ggctatggggacctgggctg

SEQ ID NO: 10 - DNA, 20 bp, Homo sapiens
aagttcctgtgatgtcaagc

SEQ ID NO: 11 - DNA, 20 bp, Homo sapiens
tcatgagcagattaaacccg

SEQ ID NO: 12 - DNA, 20 bp, Homo sapiens
gcatcctgaccgtgtccgaa

SEQ ID NO: 13 - DNA, 19 bp, Homo sapiens
gggtcagtaccagtgccag

SEQ ID NO: 14 - DNA, 17 bp, Homo sapiens
atggactggacctggag

SEQ ID NO: 15 - DNA, 22 bp, Homo sapiens
atggacatactttgttccacgc

SEQ ID NO: 16 - DNA, 22 bp, Homo sapiens
atggacacactttgctccacgc

SEQ ID NO: 17 - DNA, 21 bp, Homo sapiens
atgaaacacctgtggttcttc

SEQ ID NO: 18 - DNA, 18 bp, Homo sapiens
atgtctgtctccttcttc

SEQ ID NO: 19 - DNA, 24 bp, Homo sapiens
cgtatccgacggggaattctcaca

SEQ ID NO: 20 - DNA, 23 bp, Homo sapiens
gaggtgmarctgcagsagtcwgg

SEQ ID NO: 21 - DNA, 28 bp, Homo sapiens
cagggtgaccagggtaccttggccccag

SEQ ID NO: 22 - DNA, 24 bp, Homo sapiens
gamayygagctcacccagtctcca

SEQ ID NO: 23 - DNA, 24 bp, Homo sapiens
cgggaagatgaagacagatggtgc

SEQ ID NO: 24 - DNA, 22 bp, Homo sapiens
gtttgatctccagcttggtccc

SEQ ID NO: 25 - DNA, 21 bp, Homo sapiens
cagtctgccctgactcagcct

SEQ ID NO: 26 - DNA, 18 bp, Homo sapiens
tcctatgagctgacwcag

SEQ ID NO: 27 - DNA, 18 bp, Homo sapiens
cgtgtggccttgttggct

SEQ ID NO: 28 - DNA, 21 bp, Homo sapiens
taggacggtsascttggtccc

(19) United States
(12) Patent Application Publication
(10) Pub. No.: US 2006/0156422 A1
(43) Pub. Date: Jul. 13, 2006

(54) Methods and compositions for the generation of antibodies

(75) Inventors: Michael Alexander Dalrymple, Edinburgh (GB); Edward Jude Gallagher, Edinburgh (GB); Ming Hong Shen, Edinburgh (GB); Howard John Cooke, Edinburgh (GB)

Correspondence Address: PALMER & DODGE, LLP;KATHLEEN M. WILLIAMS, 111 HUNTINGTON AVENUE, BOSTON, MA, 02199, US

(73) Assignee: Medical Research Council

(21) Appl. No.: 11/032,668

(22) Filed: Jan. 10, 2005

(51) Int. Cl. A01K 67/027; C12N 5/16; C12N 5/06

(52) U.S. Cl. 800/6; 800/18; 435/326; 435/354

(57) ABSTRACT

The present invention relates to a novel method for the generation of monoclonal antibodies. In particular the invention relates to a novel method for the generation of monoclonal antibodies from non-human somatic transgenic animals. Uses of antibodies generated using the method of the invention are also described.

The present invention relates to a novel method for the generation of monoclonal antibodies In particular the invention relates to a novel method for the generation of monoclonal antibodies from transgenic mice transplanted with donor cells. The novel method is not a germline dependent method. Uses of antibodies generated using the method of the invention are also described.

Antibodies are key components of the body's defence mechanisms. They are proteins produced by cells of the immune system which specifically recognise target (usually foreign) molecules known. Much research has been targeted towards using the exquisite specificity of antibodies to treat a wide ranges of diseases.

Despite the early recognition of antibodies, as promising therapeutic agents, most approaches towards developing them as products have been met with a number of commercial and technical limitations. Initial efforts were aimed at the development of hybridoma cells, which are immortalized mouse antibody-secreting B cells. Such hybridoma cells are derived from normal mouse B cells that have been genetically manipulated so that they are capable of reproducing over an indefinite period of time. Such cells are cloned to produce a homogeneous population of identical cells which produce one single type of mouse antibody capable of recognizing one specific antigen. These are known as monoclonal antibodies or MAbs.

The case for exploiting antibodies as therapeutic agents is now proven and the routes for clinical development established and accepted. There is currently a rapid increase in the recognition and identification of new therapeutic targets through mechanisms such as the human genome project and thus demand for new therapeutic antibodies against novel targets is steadily growing. As a consequence, there is expected to be a continued demand for the generation of partially human, mainly human or fully human antibodies.

The first therapeutic MAb to be approved was a murine MAb, OrthoClone OKT3, launched by Johnson & Johnson in 1986, and used to help prevent the rejection of transplant organs. However, while mouse MAbs can be generated to bind to a number of antigens, they contain mouse protein sequences and tend to be recognised as foreign by the human immune system. As a result, they are quickly eliminated by the human body and have to be administered frequently. An additional problem with the use of such antibodies is that when patients are repeatedly treated with mouse antibodies, they often begin to produce antibodies that effectively neutralise the mouse antibody, a reaction referred to as a Human Anti-Mouse Antibody (“HAMA”) response. In many cases, the HAMA response prevents the mouse antibodies from having the desired therapeutic effect and may cause the patient to have an allergic reaction.

Recognising the limitations of mouse monoclonal antibodies, more recently researchers have developed a number of approaches to prevent mouse MAbs rejection by a patient's immune system. For example, improved forms of mouse antibodies, referred to as “chimaeric” and “humanised” antibodies, are genetically engineered and assembled from portions of mouse and human antibody gene fragments.

Chimaeric antibodies (e.g. Eli Lilly's ReoPro) refer to those antibodies which contain a mixture of mouse and human antibody components. Humanised antibodies (e.g. Roche's Zenapax) on the other-hand refer to antibodies which comprise binding sites from a non-human donor antibody onto a human antibody structure (CDR grafted). MAbs While such chimaeric and humanised antibodies trigger an immune response in a human recipient to a lesser degree than monoclonal antibodies of mouse origin, they still retain a varying amount of the mouse antibody protein sequence and, accordingly, may continue to trigger the HAMA response. Additionally, the humanisation process can be expensive and time consuming, requiring at least months and sometimes over a year of secondary manipulation after the initial generation of the mouse antibody. Once the humanisation process is complete, the remodelled antibody gene must then be expressed in a recombinant cell line appropriate for antibody manufacturing, adding additional time before the production of preclinical and clinical material can be initiated. In addition, the combination of mouse and human antibody gene fragments can result in a final antibody product which is different in structure from the original mouse antibody, leading to affinity and specificity problems.

Thus there remains a need in the art for a simple and cost effective method for the production of human-derived monoclonal antibodies for therapeutic use.

There are currently two main methods for making fully human MAbs:

(i) Phage display technology: the engineering of bacteriophage to display human monoclonal antibodies on their surfaces has been used to create libraries containing over 100 billion human monoclonal antibodies. The libraries are used to identify target molecules in high throughput screens. This technology, through Cambridge Antibody Technology (CAT) has given rise to Humira, the first fully-human MAb to reach the market (Wood Mackenzie Horizons, Pharmaceuticals Issue 6, January 2003. ‘Monoclonal antibodies—on the crest of a wave.’).

(ii) Germ-line transgenic mice: antibodies with fully human protein sequences can be produced using germline genetically engineered strains of mice in which mouse antibody gene expression is suppressed and functionally replaced with human antibody gene expression, while leaving the rest of the mouse immune system intact. Rather than engineering each antibody product candidate, these transgenic mice capitalise on the natural power of the mouse immune system in surveillance and affinity maturation to produce a broad repertoire of high affinity antibodies. For example, Abgenix uses transgenic mice expressing the human heavy and light Ig loci to create antibodies with fully human protein sequences. Several strains of this XenoMouse have been developed, each of which is capable of producing a different class of antibody to perform different therapeutic functions, (Green 1999). Details are described in WO 9402602 which is herein incorporated by reference. MAbs Medarex is a biopharmaceutical company developing monoclonal antibody-based therapeutics to fight cancer and other life-threatening and debilitating diseases. The company has developed a broad platform of patented technologies for antibody discovery and development, including Trans-Phage Technology and the UltiMAb Human Antibody Development System. This uses Medarex's HuMAb-Mouse, the Kirin TC (transchromosomic) Mouse which has been acquired through an exclusive partnership with Kirin Brewery Company of Japan, and the crossbred KM-Mouse.

Several problems exist with prior art methods for the generation of transgenic mice. For example, the use of yeast artificial chromosomes (YACs) as vectors to create transgenic animals expressing fully humanized antibodies, for example as used by Medarex, suffers from several short comings. One limitation of the YAC vector is the carrying capacity of insert DNA. In theory, YACs can have insert size cloning capacity in the range of 1-2 Mb; in practice the useful YACs isolated containing portions of the human Ig loci tend to have insert sizes <1 Mb. The human Ig heavy chain is approximately 1.5 Mb, the light chain loci are ˜2 Mb and 1.5 Mb, therefore individual YAC clones do not carry an intact locus. YAC vectors are also unstable and are prone to rearrangement in yeast cells, often deleting portions of the insert DNA. This is a particular problem when cloning Ig loci as they consist of many closely related gene sequences that are prone to recombination in yeast cells. For these reasons the full repertoire of human antibody types has not been produced reliably using YAC vectors (Bruggemann 2001). Another technical problem that arises when transferring YAC vectors from yeast cells to mouse ES cells by spheroplast fusion, is that often large portions of the yeast genome are co transferred into the mouse ES cells. This often has a deleterious effects on the recovered ES cell clones and hence their subsequent inability to make chimaeric mice.

The use of human whole chromosomes or chromosome fragments (hCFs) as vectors for introducing the Ig loci in to mouse cells has proven to overcome some of the drawbacks of using YACs. The larger carrying capacity of the chromosome vectors can incorporate the whole Ig loci, so in theory the complete repertoire of human antibodies can be produced by the engineered mice. This approach has been adopted by the Kirin Company (Tomizuka et al 2000). However the human chromosomes or their fragments are not as stable as YAC vectors, which can integrate into the mouse genome. The mice produced so far have demonstrated various levels of trans-loci retention at the somatic level and also in the ability of the mice to transmit to the next generation. The ability of mouse cells to retain a human chromosome is dependent upon the chromosome type. For chromosome fragments, there would also appear to be a correlation between decreased fragment size and increased mitotic stability (Tomizuka et al 1997). The Kirin company have reported a 0.1% loss per cell division for a human chromosome fragment (hCF) of chromosome 14 in ES cells. However, for chromosome 2, that loss was 3.2%. This instability translates to actual retention frequency of 78+/−13% for hCF 14 and 30+/−11% for hCF 2 of the resulting mice made from these transgenic ES cell lines (Ishida et al 2002). This instability not only lowers the efficiency with which these mice can pass the trans-loci to the next generation but also leads to mosaic pattern of expression within the offspring and even fewer cells retaining the trans-loci. The problem of hCF transmission through the germline is multiplied when an animal has to inherit at least 2 chromosome fragments to produce cells capable of human antibody production. The result of this loss of Ig loci from the ES derived B cells is that they are unable to generate human antibodies.

Thus there still remains a need in the art for a simple and effective method for the production of both fully and partially human monoclonal antibodies.

### SUMMARY OF INVENTION

The present inventors have devised a new, non-germline dependent, method for the production of both fully and partially human monoclonal antibodies. The inventors consider that such a method overcomes the problems exhibited by prior art methods for their production.

Thus in a first aspect the present invention provides a method for the generation of a heterologous immunoglobulin molecule from a non-human somatic transgenic animal which method comprises the steps of:
(a) Creating one or more non-human somatic transgenic animal/s (i) by transfecting suitable donor cells with nucleic acid encoding a heterologous immunoglobulin molecule, (ii) transplanting said cells into a host animal; wherein the native immune system of those recipient host animal/s is suppressed and/or functionally inactivated (thus generating a non-human somatic transgenic animal); and
(b) Expressing one or more heterologous immunoglobulin molecules from one or more cells of those host animals treated according to step (a).

According to the above aspect of the invention, preferably the method comprises the optional additional steps of: differentiation of donor cells into cell types more suitable for transplantation or commitment of donor cells to a B cell developmental fate prior to transplantation into recipient/host animals and the enrichment of differentiated donor cells. One or more of these steps may be performed after step (a) recited above. For the avoidance of any doubt the term ‘differentiation’ according to the present invention includes within its scope those cells which are committed to a particular cell fate but not yet specialised.

According to the method of the invention, ‘cell types more suitable for transplantation into host/recipient animals’ and donor cells ‘committed to a B-cell developmental fate’ preferably comprise any of those cells or combinations thereof in the list consisting of the following: haemopoetic stem cells, pre-B cells, pre-pro B-cells and pro B-cells. Those skilled in the art will appreciate that such cells are characterised by the presence of certain surface markers.

In addition, advantageously, further steps are included according to the method of the invention. Importantly, step (b) recited above, that is the step of expressing one or more heterologous immunoglobulin molecules from one or more cells of those somatic-transgenic animals generated according to step (a) comprises the establishment of hybridomas/hybridoma cell lines from one or more of those recipient animals and the expression of heterologous immunoglobulin molecules therefrom.

According to the above aspect of the invention, preferably the animal is a rodent or of porcine origin. More preferably it is a mouse.

According to the method of the invention described herein, preferably the donor cells for transfection of nucleic acid encoding a heterologous immunoglobulin molecule of interest are embryonic stem cells (ES cells). Other suitable cells include but are not limited to embryonic germ (EG cells), bone marrow cells, B-cells, multipotent progenitor cells, MAPCs derived from adult bone marrow and other pluripotent cells derived from somatic tissue. Other suitable cell types for use according to the methods of the present invention are described in the following references which are herein incorporated by reference: Yuehua j. et al Pluripotency of mesenchymal stem cells derived from adult marrow. Nature 2002. Léobon B, Garcin I, Menasché P, Vilquin J T, Audinat E, Charpak S. Myoblasts transplanted into rat infarcted myocardium are functionally isolated from their host. Proc Natl Acad Sci USA; Chen J C, Goldhamer D J. Skeletal muscle stem cells. Reprod Biol Endocrinol. 2003; Li A, Pouliot N, Redvers R, Kaur P. Extensive tissue-regenerative capacity of neonatal human keratinocyte stem cells and their progeny. J Clin Invest. 2004; Prockop D J, Gregory C A, Spees J L. One strategy for cell and gene therapy: Harnessing the power of adult stem cells to repair tissues. Proc Natl Acad Sci USA. 2003; Fodor W L. Tissue engineering and cell based therapies, from the bench to the clinic: The potential to replace, repair and regenerate. Reprod Biol Endocrinol. 2003. Those skilled in the art will appreciate that this list is not intended to be exhaustive.

Transfected donor cells may be subjected to in vitro differentiation and cell enrichment using any suitable method for example FACS/MACS sorting and/or affinity based column purification. Those skilled in the art will appreciate that this list is not intended to be exhaustive. Such techniques enrich for particular cell types prior to transplantation into a host animal. For example in the case where the donor cells are embryonic stem cells (ES cells) then such cells may be treated such that they differentiate into haemopoietic stem cells (HSCs), pre-pro B cells, pro B cells, pre-B cells or mixtures thereof prior to transplantation. For the avoidance of any doubt any as herein defined, any cell type which has the capacity to dfferentiate to from a B-cell (including haemopoietic stem cells) is included within the scope of the term ‘immature B cell’ and will be referred to herein as such. Such treatments ensure the transplanted cells specifically colonise the B-cells compartments of the donor animal. Further these differentiated cells may then be sorted and enriched for a particular cell type. Thus in this case, differentiated cells may be sorted and those cells which have differentiated into HSCs selected for transplantation into recipient animals according to the methods herein described.

In an alternative embodiment of the method of the invention, non-differentiated cells are transplanted into hosts, and some of those cells may correctly colonise the intended organ niche. Details of suitable techniques used in this aspect of the invention are described in Bjorklund et al. Embryonic stem cells develop into functional dopaminergic neurons after transplantation in a Parkinson rat model. Proc Natl Acad Sci USA. 2002

The problem with such an approach is that injected non-differentiated cells may aggregate forming clumps and potentially cancerous cell growths, such as teratocarcinomas, with only a small percentage of the cells correctly grafting and differentiating. The present inventors consider that these problems may be overcome using a variety of techniques:
(i) If the ES cells are delivered at a reduced cell density directly to the site of engraftment then the inventors consider that it is possible to have an efficient and functional route of cell delivery. Reducing the cell numbers injected into any one site may also avoid the problems of aggregation. Furthermore, injection of ES cells into the site of engraftment would alleviate the problems of homing to the correct site and provide the necessary environmental cues for correct differentiation.
(ii) Alternatively or in addition, the donor cells may be engineered to express a cell surface protein that improves the ability of the cell to associate with its target, again improving the low efficiency of this approach.
(iii) Alternatively or in addition to (i) and (ii) above, donor cells may be exposed, prior to injection, with a particular growth factor or cytokine that would trigger or enhance the differentation of donor cells, in partciular the differentation of ES cells to haematopoietic cells.

In addition, advantageously donor cells (for example ES cells) have their endogenous Ig locus inactivated, using the techniques of gene targeting. For the avoidance of any doubt this should not be confused with the functional inactivation of host animals' endogenous Ig locus.

Any suitable method for the transfection of nucleic acid encoding a heterologous an immunoglobulin molecule into suitable recipient cells may be employed. Suitable methods include but are not limited to any one or more of the following: electroporation, micro-injection, microcell or whole cell fusion and liposomal transfection. Those skilled in the art will appreciate that this list is not intended to be exhaustive. Details of these procedures are provided the detailed description of the invention.

Donor cells are then transplanted into a host animal (for example an immune-compromised mouse) using methods known to those skilled in the art and described in the detailed description of the invention. After subsequent immunization the splenocytes (from which B-cells are derived) are removed from the host animal for the generation of hybridomas and the production of heterologous immunoglobulin molecules, preferably human monoclonal antibodies. Methods for the production of hybridomas will be familiar to those skilled in the art and are described in the detailed description of the invention.

According to the method described herein the term ‘heterologous’ (immunoglobulin) refers to an immunoglobulin molecule which is not in its native environment. Thus in the present case, it refers to a protein present within a species of organism other than the species of organism which it is derived from/originates from. Thus in the case where the transgenic animal is a mouse, then a protein which is of mouse origin and is expressed within those mouse cells then such a protein would not be heterologous. In this case it would be termed a homologous protein. However, in the case where the transgenic animal is a mouse and the expressed protein is derived from or is identical to a human protein then such a human protein expressed within transgenic mice is referred to as a heterologous protein herein.

In a preferred embodiment of the above aspect of the invention, the heterologous immunoglobulin molecule is a human immunoglobulin molecule. More advantageously, the heterologous immunoglobulin molecule expressed according to the method of the invention is an antibody molecule, preferably a human antibody molecule. The antibody can be, for example, a monoclonal antibody, polyclonal antibody, dAb, scFv or Fab. Antibody molecules suitable for expression using the method of the invention will be familiar to those skilled in the art and are described in the detailed description of the invention.

According to the Method of the invention, the term ‘transgenic animal’ refers to an animal which comprises one or more nucleic acid elements which are heterologous, that is nucleic acid elements which are other than those of the host animal. Accordingly, the term ‘somatic transgenic animal’ refers to a transgenic animal in which the transgene is inserted somatically, that is generally in the young or adult animal as opposed to the insertion of one or more transgenes in the germline prior to the generation of young or adult animals. This latter method for the preparation of ‘germ-line transgenic animals’ is the method which has been used for the production of antibody molecules for therapeutic use prior to the filing of the present application. Methods for the generation of somatic transgenic animals are described in the detailed description of the invention.

For the avoidance of any doubt according to the method described herein, ‘recipient host animals’ for transplantation with ‘suitable donor cells’ according to the method described herein do not include within their scope embryos. That is, ‘host-animals’ used for the generation of ‘somatic-transgenic animals’ according to the invention are young or adult animals. In a preferred embodiment of the method of the invention, they are young or adult mice, or other similar rodents.

According to the above aspect of the invention, the term ‘immune system’ (of an animal) refers to one or more elements of the innate and/or acquired immune system of that animal. Furthermore, the term the ‘native immune system’ (of an animal) refers to the immune system which is inherent to the animal upon its development and which is generated from the germ-line of that animal. Those skilled in the art will appreciate that such a ‘native immune system’ has innate and acquired elements. The term ‘suppressing the native immune system’ (of an animal) refers to the suppression of one or more elements of the inherent innate and/or acquired immune system of that animal. Advantageously the term ‘suppression of the native immune system of an animal’ refers to the suppression of one or more elements of the inherent innate and/or acquired immune system of an animal such that a foreign protein introduced into that animal is not eradicated as a consequence of an immune reaction raised in response to its presence in that animal. Advantageously, the term ‘suppression’ means the suppression of the native immune system (of an animal) as defined herein such that 10%, 20%, 30% 40%, 50 or 60% of a foreign protein introduced into that animal is not eradicated as a result of the action of the host animals innate and/or acquired immune system against that protein. More advantageously, the term ‘suppression’ refers to the suppression of the native innate and/or acquired immune system of the host animal such that 70%, 80%, 90% of a foreign protein introduced into that animal is not eradicated as a result of the host animals innate and/or acquired immune system acting against it. Most advantageously, the term ‘suppression’ refers to the suppression of the native innate and/or acquired immune system of the host animal such that 92, 94, 96, 98 or 100% of a foreign protein introduced into that animal is not eradicated as a result of the host animals innate and/or acquired immune system acting against it.

An essential feature of the invention described herein is that the method is entirely somatic and will not involve transmission through the germ cells. Importantly, this approach does not require the creation or maintenance of germ line transmissible transgenic animals. Accordingly, the somatic-transgenic animals generated according to the methods of the invention cannot produce offspring which inherit the capability of expressing heterologous immunoglobulin molecules as herein defined.

A further feature of the invention described herein is that the method of the invention generates a transient resource for the production of heterologous immunoglobulin molecules (HIs) from cells, preferably B-cells which can be immortalised by standard methods.

In one aspect, there is provided a method of producing an antibody display library, the method comprising: (a) introducing an antigen into a non-human somatic transgenic animal generated according to the methods described herein; (b) isolating a population of nucleic acids encoding one or more antibody chains from one or more lymphatic cells of that somatic transgenic animal; and (c) forming a library from those antibody chains.

Also encompassed are hybridoma cells expressing human monoclonal antibody molecules, wherein the hybridoma cells are generated from non-human somatic-transgenic animals obtained using the methods described herein.

Also encompassed is a heterologous immunoglobulin molecule obtained by the method described herein.

Also encompassed is a non-human somatic transgenic animal obtained using a method described herein, comprising nucleic acid encoding one or more heterologous immunoglobulin molecules, wherein the native/endogenous immune system of that animal is suppressed and/or functionally inactivated.

Also encompassed is a non-human somatic transgenic animal comprising nucleic acid encoding one or more heterologous immunoglobulin molecules, wherein the native/endogenous immune system of that animal is suppressed and/or functionally inactivated. The heterologous immunoglobulin molecule can be, for example, an antibody molecule which is at least partially human, preferably fully human.

Also encompassed are pleuripotent cells which are committed to a B-cell fate and which comprise nucleic encoding one or more heterologous immunoglobulin molecules. In one embodiment, the heterologous immunoglobulin molecules are antibody molecules. Such pleuripotent cells can be used as donor cells in the methods described herein.

The creation of ‘somatic transgenic animals’ using the method of the invention relies upon the transplantation of suitable donor cells described herein expressing the human heterologous immunoglobulin molecule/s (ES-HI) into immuno-compromised host animals.

Suitable immuno-compromised animals for use according to the method of the invention include, for example, but are not limited to the SCID or RAG deficient mouse. Such mice are described in WO9505736A1; U.S. Pat. No. 23,182,671A; WO09305796A1 and U.S. Pat. No. 5,859,307 These patent documents are herein incorporated by reference. RAG and SCID mice are described more generally in Dorshkind et al. Functional status of cells from lymphoid and myeloid tissues in mice with severe combined immunodeficiency disease. J. Immunol. 1984; Mombaerts et al. RAG-1-deficient mice have no mature B and T lymphocytes. Cell. 1992; Shinkai et al. RAG-2-deficient mice lack mature lymphocytes owing to inability to initiate V(D)J rearrangement. Cell. 1992. Other suitable immuno-compromised mice include a lethally irradiated mice. Those skilled in the art will be aware of other suitable immuno-compromised animals and will appreciate that the list provided above is not intended to be limiting.

In a further aspect the invention provides immunoglobulin molecule obtained by the method of the invention.

Advantageously, the immunoglobulin molecule is an antibody molecule, more preferably a human antibody molecule.

Human antibody molecules produced according to the methods of the invention may be fully/entirely human, that is where the entire antibody is encoded by nucleic acid of human origin and/or the primary amino acid sequence of the antibody is identical to that of an antibody of human origin.

Alternatively, an antibody produced according to the methods of the invention may be ‘partially human’. That is, an antibody produced according to the method of the invention may have a portion (at least 2, 3, 4, 5, 6, 8, 10%, 20%, 30%, 40%, 50%, 60% 70%, 80%, 90% or at least 95% but less than 100%) which is of human origin or is identical in amino acid sequence to an antibody of human origin.

In yet a further aspect the invention provides a somatic transgenic animal comprising nucleic acid encoding one or more heterologous immunoglobulin molecules, wherein the native immune system of that animal is suppressed.

Advantageously, the somatic transgenic animal according to the above aspect of the invention is a rodent, preferably a mouse. However, other somatic transgenic animals may be produced using the method of the present invention. Such animals include non-human primates, such as chimpanzee, bovine, ovine, porcine, shark and camellia species, and other members of the rodent family, e.g. rat, as well as rabbit and guinea pig. Particular preferred animals are mouse, rat, rabbit and guinea pig, most preferably mouse.

More advantageously the somatic transgenic animal is a mouse and the heterologous immunoglobulin molecule is preferably a human immunoglobulin molecule, more preferably still a human antibody molecule.

Methods for the suppression of the native immune system of that animal are described above and in the detailed description of the invention.

The inventors consider that heterologous proteins, in particular monoclonal antibodies produced using the method of the invention will have great therapeutic value.

Thus in a further aspect the present invention provides the use of a human immunoglobulin molecule obtained using the method of the invention in the treatment of disease.

In yet a further aspect the invention provides a method for the treatment of one or more diseases in a patient comprising the step of administering to that patient in need of such treatment an effective amount of a human immunoglobulin molecule obtained using the method of the invention.

In a further aspect still the invention provides a method for the generation of polyclonal antibodies from a somatic transgenic animal according to the invention which method comprises immunising that somatic transgenic animal with an antigen and isolating the polyclonal antiserum from that animal.

In yet a further aspect the invention provides, hybridoma cells expressing human monoclonal antibody molecules, wherein the hybridoma cells are generated from somatic-transgenic animals obtained using the method of the invention.

Definitions.

‘Immunoglobulin’ This refers to a family of polypeptides which retain the immunoglobulin fold characteristic of antibody molecules, which contains two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). Preferably, the present invention relates to antibodies.

‘Antibody ’: An antibody (for example IgG, IgM, IgA, IgD or IgE) or fragments such as a FAb, F(Ab′)₂, Fv, disulphide linked Fv, scFv, diabody, dual-specific antibodies and single domain antibodies (dAbs) whether derived from any species naturally producing an antibody, or created by recombinant DNA technology.

Human antibody molecules produced according to the methods of the invention may be ‘fully/entirely human’, that is where the entire antibody is encoded by nucleic acid of human origin and/or the primary amino acid sequence of the antibody is identical to that of an antibody of human origin.

Alternatively, an antibody produced according to the methods of the invention may be ‘partially human’. That is, an antibody produced according to the method of the invention may have a portion (at least 2, 3, 4, 5, 6, 8, 10%, 20%, 30%, 40%, 50%, 60% 70%, 80%, 90% or at least 95% but less than 100%) which is of human origin or is identical in amino acid sequence to an antibody of human origin.

‘Epitope’ A unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation.

‘Antigen’: A ligand that is bound by one or more antibody molecules. A specific antigen comprises one or more epitopes. It may be a polypeptide, protein, nucleic acid or other molecule.

‘Transgenic animal’ according to the present invention refers to an animal which comprises one or more large human DNA fragments, such as genomic DNA and/or chromosme fragments and/or whole chrmosomes which are heterologous, that is nucleic acid elements which are other than those of the host animal. Vector suitable for the generation of such translocus animals according to the method of the invention include YAC or BAC vectors which are capable of integrating large pieces of nucleic acid with an upper limit of 1-2 Mb and 100-200 Kb respectively.

Accordingly, the term ‘somatic transgenic animal’ refers to a transgenic animal in which the Ig translocus is inserted somatically, that is generally in the young or adult animal as opposed to the insertion of one or more transgenes in the germline prior to the generation of young or adult aminals. Somatic transgenic animals according to the invention consist of cell types of two or more distinct genetic origins ie non-self, that form an individual animal. The creation of that animal does not involve the donor cells contributing to the germline of the host animal. Therefore, any offspring generated from a somatic transgenic animal according to the invention will be derived from the germ cells of the host and not the donor cells of the somatic transgenic animal. The donor cells are typically of ES cell origin. The donor cells are in addition typically transgenic for the gene or genes of interest and in the case of this invention preferably the human Ig loci.

Methods for the generation of somatic transgenic animals are described in the detailed description of the invention. Advantageously, the somatic transgenic animal according to the above aspect of the invention is a rodent, preferably a mouse. However, other somatic transgenic animals may be produced using the method of the present invention. Somatic transgenic animals according to the present invention include non-human primates, such as chimpanzee, bovine, ovine, and porcine species, other members of the rodent family, e.g. rat, as well as rabbit and guinea pig. Particular preferred animals are mouse, rat, rabbit and guinea pig, most preferably mouse.

As referred to herein the term ‘recipient/host animals’ for transplantation with ‘suitable donor cells’ and resulting in the generation of somatic transgenic mice according to the method described herein does not include within its scope embryos. That is, ‘host-animals’ used for the generation of ‘somatic-transgenic animals’ according to the invention are young or adult animals. In a preferred embodiment of the method of the invention, they are young or adult mice, or other similar rodents.

As referred to herein, the term ‘endogenous Ig locus knock-out mice’ refers to mice in which the endogenous immunoglobulin locus has been inactivated. Further, according to the methods of the present invention, advantageously the term ‘host animals in which the endogenous/native immune system has been suppressed and/or functionally inactivated’ refers to those host animals in which the endogenous Ig locus has been inactivated by gene targeting.

‘Heterologous’ (immunoglobulin molecule) refers to an immunoglobulin molecule which is expressed in an environment which is not its native environment Thus in the present case, it refers to a immunoglobulin molecule expressed within a species of organism other than the species of organism which it is derived from/originates from. Thus, in the case where the transgenic animal is a mouse, then an immunoglobulin molecule of mouse origin expressed within those mouse cells would not be a heterolgous protein it would be referred to herein as a homologous protein. However, in the case where the transgenic animal is a mouse and the expressed immunoglobulin molecule is derived from or is identical to a human immunoglobulin molecule then such a human immunoglobulin molecule expressed within a transgenic mice is referred to as a heterologous immunoglobulin molecule herein.

The term ‘immune system’ (of an animal) refers to one or more elements of the innate and/or acquired immune system of that animal. Furthermore, the term the ‘native immune system’ (of an animal) refers to the immune system which is inherent to the animal upon its development and which is generated from the germ-line of that animal. Those skilled in the art will appreciate that such a ‘native immune system’ has innate and acquired elements. The term ‘suppressing the native immune system’ (of an animal) refers to the suppression of one or more elements of the inherent innate and/or acquired immune system of that animal. Advantageously the term ‘suppression of the native immune system of an animal’ refers to the suppression of one or more elements of the inherent innate and/or acquired immune system of an animal such that a foreign protein introduced into that animal is not eradicated as a consequence of an immune reaction raised in response to its presence in that animal.

‘ES-hlg’ as used herein refers to mouse ES cells which have been modified to carry human DNA sequences of the Ig loci.

Vector—Vectors suitable for the generation of transgenic animals according to the present invention include any vectors which are capable of carrying and replicating large pieces of DNA. Such vectors include YAC or BAC vectors, chromosome fragments, whole chromosomes, episomal constructs and viral vectors, particularly Epstein-Barr virus (EBV) derived vectors. Those skilled in the art will appreciate that this list is not intended to be exhaustive.

Differentiation: As referred to herein the term ‘differentiation’ describes the process whereby a cell (preferably a stem cell or stem cell like cell as described herein) becomes specialised. Cell specialisation may include the expression of particular cell surface markers. In addition the term ‘differentiation’ includes within its scope those cells which are ‘committed’ to a particular developmental cell fate but have not yet begun the process of cell differentation. According to the method of the present invention preferably donor cells are used which are pleuripotent and which are committed to a B-cell fate or are both committed and differentiated to a B-cell fate are used. Preferably donor cells for use according to the method of the invention are any one or more of those cells selected from the group consisting of: embryonic stem cells (ES cells) including ES like cells as defined herein, embryonic germ (EG cells), bone marrow cells, multipotent progenitor cells, MAPCs derived from adult bone marrow and other pluripotent cells derived from somatic tissue.

Stem cell: A cell that has the ability to continuously divide, (is capable of self renewal), and can differentiate into various other cell types. This includes embryonic cell types which are pluripotent and can in theory develop into all cells types, as well as adult stem cells which are more limited in their potential to differentiate into other cell types (typically one or two cell types). According to the present invention, the term ‘stem-cell’ includes within its scope stem-cell like cells (stem-like cells) as defined herein.

Stem-like cell: As referred to herein means any cell type that possesses the capacity of self renewal and the ability to differentiate as herein defined. The capacity of stem-like cells for self renewal is often finite in contrast to the theoretical infinite self renewal capacity of embryonic stem cells. Stem-like cells have a limited capacity for self renewal but are able to differentiate to cell types capable of making immunoglobulin molecules, in particular antibody molecules.

According to the method of the invention, ‘cell types more suitable for transplantation into host/recipient animals’ and donor cells ‘committed to a B-cell developmental fate’ preferably comprise any of those cells or combinations thereof in the list consisting of the following: haemopoetic stem cells, pre-B cells, pre-pro B-cells and pro B-cells. Those skilled in the art will appreciate that such cells are characterised by the presence of certain surface markers. As herein defined, any cell type which has the capacity to dfferentiate into a B-cell (including haemopoietic stem cells) is included within the scope of the term ‘immature B cell’ and will be referred to herein as such.

### BRIEF DESCRIPTION OF THE FIGURES

FIG. 1: Schematic representation of an embodiment of the methods described herein.

FIG. 2: Schematic representation of the inactivation of endogenous Heavy chain locus of the mouse by a 2-step gene targeting strategy that employs the expression of Cre recombinase to delete the locus intervening the engineered lox P sites. Step 1 required the integration of a targeting construct consisting of homology to the 5′ end of the Ig Heavy locus, selectable markers for the neomycin and HPRT genes, and a loxP site. Recovery of recombinant clones was by G418 selection and targeted clones were identified as detailed in the methods of Example 3. Step 2 required the targeted clones to be re-targeted with a second construct. The second construct consisted of homology to the 3′ end of the Ig Heavy gene sequence, a hygromycin selectable marker a HPRT selectable marker along with a loxP site were placed within the arms of homology in the order indicated. Identified clones that have been correctly targeted, for a second time, are transiently exposed to Cre recombinase and selected in 6-TG as detailed in the methods of Example 3. Deletion of the DNA sequence intervening the loxP sites produces ES cell lines that are hemizygous for the desired deletion i.e. lacking one allele of the Heavy chain locus.

FIG. 3: Schematic representation of the inactivation of the endogenous Kappa locus of the mouse by a 2-step gene targeting strategy that employed the expression of Cre recombinase to delete the locus intervening the engineered lox P sites. Step 1 required the integration of a targeting construct consisting of homology to the 5′ end of the Ig Kappa locus, selectable markers for the neomycin and HPRT genes, and a loxP site. Recovery of recombinant clones was by G418 selection and targeted clones were identified as detailed in the methods of Example 3. Step 2 required the targeted clones to be re-targeted with a second construct. The second construct consisted of homology to the 3′ end of the Ig gene sequence, a hygromycin selectable marker a HPRT selectable marker along with a loxP site were placed within the arms of homology in the order indicated. Identified clones that have been correctly targeted, for a second time, are transiently exposed to Cre recombinase and selected in 6-TG as detailed in the methods of Example 3. Deletion of the DNA sequence intervening the loxP sites produces ES cell lines that are hemizygous for the desired deletion i.e. lacking one allele of the Kappa locus.

FIG. 4: Schematic representation of the inactivation of the endogenous Lambda locus of the mouse by a 2-step gene targeting strategy that employed the expression of Cre recombinase to delete the locus intervening the engineered lox P sites. Step 1 required the integration of a targeting construct consisting of homology to the 5′ end of the Ig Lambda locus, selectable markers for the neomycin and HPRT genes, and a loxP site. Recovery of recombinant clones was by G418 selection and targeted clones were identified as detailed in the methods of Example 3. Step 2 required the targeted clones to be re-targeted with a second construct. The second construct consisted of homology to the 3′ end of the Ig Lambda gene sequence, a hygromycin selectable marker a HPRT selectable marker along with a loxP site were placed within the arms of homology in the order indicated. Identified clones that have been correctly targeted, for a second time, are transiently exposed to Cre recombinase and selected in 6-TG as detailed in the methods of Example 3. Deletion of the DNA sequence intervening the loxP sites produces ES cell lines that are hemizygous for the desired deletion i.e. lacking one allele of the Lambda locus.

### DETAILED DESCRIPTION OF THE INVENTION

General Techniques

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

The Structure and Generation of Antibodies.

The basic structure of all immunoglobulins is based upon a unit consisting of two light polypeptide chains and two heavy polypeptide chains. Each light chain comprises two regions known as the variable light chain region and the constant light chain region. Similarly, the immunoglobulin heavy chain comprises two regions designated the variable heavy chain region and the constant heavy chain region.

The constant region for the heavy or light chain is encoded by genomic sequences referred to as heavy or light constant region gene (C_{M}) segments. The use of a particular heavy chain gene segment defines the class of immunoglobulin.

For example, in humans, the μ constant region gene segments define the IgM class of antibody whereas the use of a constant region gene segment defines the IgG class of antibodies as well as the IgG subclasses IgG1 through IgG4.

Similarly, the use of α1 or α2 constant region gene segment defines the IgA class of antibodies as well as the subclasses, IgA1 and IgA2. The δ and ε constant region gene segments define the IgD and IgE antibody classes, respectively.

The variable regions of the heavy and light immunoglobulin chains together contain the antigen binding domain of the antibody. Because of the need for diversity in this region of the antibody to permit binding to a wide range of antigens, the DNA encoding the initial or primary repertoire variable region comprises a number of different DNA segments derived from families of specific variable region gene segments. In the case of the light chain variable region, such families comprise variable (V) gene segments and joining (J) gene segments. Thus, the initial variable region of the light chain is encoded by one V gene segment and one J gene segment each selected from the family of V and J gene segments contained in the genomic DNA of the organism. In the case of the heavy chain variable region, the DNA encoding the initial or primary repertoire variable region of the heavy chain comprises one heavy chain V gene segment, one heavy chain diversity (D) gene segment and one J gene segment, each selected from the appropriate V, D and J families of immunoglobulin gene segments in genomic DNA.

In order to increase the diversity of sequences that contribute to forming antibody binding sites, it is preferable that a heavy chain transgene include cis-acting sequences that support functional V-D-J rearrangement that can incorporate all or part of a D region gene sequence in a rearranged V-D-J gene sequence. Typically, at least about 1 percent of expressed transgene-encoded heavy chains (or mRNAs) include recognizable D region sequences in the V region. Preferably, at least about 10 percent of transgene-encoded V regions include recognizable D region sequences, more preferably at least about 30 percent, and most preferably more than 50 percent include recognizable D region sequences.

A recognizable D region sequence is generally at least about eight consecutive nucleotides corresponding to a sequence present in a D region gene segment of a heavy chain transgene and/or the amino acid sequence encoded by such D region nucleotide sequence. For example, if a transgene includes the D region gene DHQ52, a transgene-encoded mRNA containing the sequence 5′-TAACTGGG-3′ located in the V region between a V gene segment sequence and a J gene segment sequence is recognizable as containing a D region sequence, specifically a DHQ52 sequence. Similarly, for example, if a transgene includes the D region gene DHQ52, a transgene encoded heavy chain polypeptide containing the amino acid sequence -DAF- located in the V region between a V gene segment amino acid sequence and a J gene segment amino acid sequence may be recognizable as containing a D region sequence. However, since D region segments may be incorporated in VDJ joining to various extents and in various reading frames, a comparison of the D region area of a heavy chain variable region to the D region segments present in the transgene is necessary to determine the incorporation of particular D segments. Moreover, potential exonuclease digestion during recombination may lead to imprecise V-D and D-J joints during V-D-J recombination.

The term “substantial similarity” denotes a characteristic of an polypeptide sequence, wherein the polypeptide sequence has at least 80, 82, 84, 86, 90, 92, 94, more preferably 96, 98 or even 99% percent identity to a reference sequence. The percentage of sequence similarity is calculated by scoring identical amino acids or positional conservative amino acid substitutions as similar. A positional conservative amino acid substitution is one that can result from a single nucleotide substitution; a first amino acid is replaced by a second amino acid where a codon for the first amino acid and a codon for the second amino acid can differ by a single nucleotide substitution.

The Primary Repertoire.

The process for generating DNA encoding the heavy and light chain immunoglobulin genes occurs primarily in developing B-cells. Prior to the joining of various immunoglobulin gene segments, the V, D, J and constant (C) gene segments are found, for the most part, in clusters of V, D, J and C gene segments in the precursors of primary repertoire B-cells. Generally, all of the gene segments for a heavy or light chain are located in relatively close proximity on a single chromosome. Such genomic DNA prior to recombination of the various immunoglobulin gene segments is referred to herein as “unrearranged” genomic DNA.

During B-cell differentiation, one of each of the appropriate family members of the V, D, J (or only V and J in the case of light chain genes) gene segments are recombined to form functionally rearranged heavy and light immunoglobulin genes. Such functional rearrangement is of the variable region segments to form DNA encoding a functional variable region. This gene segment rearrangement process appears to be sequential.

First, heavy chain D-to-J joints are made, followed by heavy chain V-to-DJ joints and light chain V-to-J joints. The DNA encoding this initial form of a functional variable region in a light and/or heavy chain is referred to as “functionally rearranged DNA” or “rearranged DNA”. In the case of the heavy chain, such DNA is referred to as “rearranged heavy chain DNA” and in the case of the light chain, such DNA is referred to as “rearranged light chain DNA”. Similar language is used to describe the functional rearrangement of the transgenes of the invention.

The recombination of variable region gene segments to form functional heavy and light chain variable regions is mediated by recombination signal sequences (RSS's) that flank recombinationally competent V, D and J segments. RSS's necessary and sufficient to direct recombination, comprise a dyad-symmetric heptamer, an AT-rich nonamer and an intervening spacer region of either 12 or 23 base pairs. These signals are conserved among the different loci and species that carry out D-J (or V-J) recombination and are functionally interchangeable. See Oettinger, et al. (1990), Science, 248, 1517-1523 and references cited therein. The heptamer comprises the sequence CACAGTG or its analogue followed by a spacer of unconserved sequence and then a nonamer having the sequence ACAAAAACC or its analogue. These sequences are found on the J, or downstream side, of each V and D gene segment.

Immediately preceding the germline D and J segments are again two recombination signal sequences, first the nonamer and then the heptamer again separated by an unconserved sequence. The heptameric and nonameric sequences following a V_{L}, V_{μ} or D segment are complementary to those preceding the J_{L}, D or J_{μ} segments with which they recombine. The spacers between the heptameric and nonameric sequences are either 12 base pairs long or between 22 and 24 base pairs long.

In addition to the rearrangement of V, D and J segments, further diversity is generated in the primary repertoire of immunoglobulin heavy and light chain by way of variable recombination between the V and J segments in the light chain and between the D and J segments of the heavy chain. Such variable recombination is generated by variation in the exact place at which such segments are joined. Such variation in the light chain typically occurs within the last codon of the V gene segment and the first codon of the J segment. Similar imprecision in joining occurs on the heavy chain chromosome between the D and J segments and may extend over as many as 10 nucleotides. Furthermore, several nucleotides may be inserted between the D and J and between the V_{μ} and D gene segments which are not encoded by genomic DNA. The addition of these nucleotides is known as N-region diversity.

After VJ and/or VDJ rearrangement, transcription of the rearranged variable region and one or more constant region gene segments located downstream from the rearranged variable region produces a primary RNA transcript which upon appropriate RNA splicing results in an mRNA which encodes a full length heavy or light immunoglobulin chain. Such heavy and light chains include a leader signal sequence to effect secretion through and/or insertion of the immunoglobulin into the transmembrane region of the B-cell. The DNA encoding this signal sequence is contained within the first exon of the V segment used to form the variable region of the heavy or light immunoglobulin chain. Appropriate regulatory sequences are also present in the mRNA to control translation of the mRNA to produce the encoded heavy and light immunoglobulin polypeptides which upon proper association with each other form an antibody molecule.

The net effect of such rearrangements in the variable region gene segments and the variable recombination which may occur during such joining, is the production of a primary antibody repertoire. Generally, each B-cell which has differentiated to this stage, produces a single primary repertoire antibody. During this differentiation process, cellular events occur which suppress the functional rearrangement of gene segments other than those contained within the functionally rearranged Ig gene. The process by which diploid B-cells maintain such mono-specificity is termed allelic exclusion.

Preparation of Somatic Transgenic Animals According to the Invention.

In a first aspect the present invention provides a method for the generation of a heterologous immunoglobulin molecule from a non-human somatic transgenic animal which method comprises the steps of:
(a) Creating one or more non-human somatic transgenic animal/s (i) by transfecting suitable donor cells with nucleic acid encoding a heterologous immunoglobulin molecule, (ii) transplanting said cells into a host animal; wherein the native immune system of those recipient host animal/s is suppressed and/or functionally inactivated (thus generating a non-human somatic transgenic animal); and
(b) Expressing one or more heterologous immunoglobulin molecules from one or more cells of those host animals treated according to step (a).
   Donor Cells

The present invention has been described with the main resource of donor cells as embryonic stem cells (ES cells) (ES-hIg) including ES-like cells. Although ES cells are the preferred cell line of choice it is possible to use other pluripotent cells such as embryonic germ cells (EG cells) or embryonic carcinoma cells (EC cells) or indeed any somatic stem cell or fused cell that is capable of re-colonising the immune system or bone marrow. Cells from a variety of tissues types can successfully re-colonise tissue types that are traditionally of a different lineage by a process known as transdifferentiation. A possible alternative to the ES cell procedure outlined is to use bone marrow derived cells. The manipulation of the stem-like or repopulating cells from the heterogeneous population of BM cells can be used instead of ES cells to create chimaeric mice capable of human antibody production. In this approach, first donor bone marrow cells are isolated, transfected with human Ig loci and then transplanted back into the mouse as outlined by the somatic transgenic approach. The direct manipulation of bone marrow cells has the added advantage of creating a population of donor cells with an increased capacity, relative to ES derived cells, to reconstitute the immune system and produce functional B cells.

Vectors for Introducing Immunoglobulin Loci into Donor Cells.

Those skilled in the art will appreciate that vectors for use according to the present invention must be capable of carrying large stretches of nucleic acid so that they are capable of facilitating the transport of one or more entire immunoglobulin loci into the donor cell of choice. Thus, vectors suitable for the generation of transgenic animals according to the present invention include any vectors which are capable of carrying and replicating large pieces of DNA. Such vectors include YAC or BAC vectors, chromosome fragments, whole chromosomes, episomal constructs and viral vectors, particularly Epstein-Barr virus (EBV) derived vectors (ref¹). Those skilled in the art will appreciate that this list is not intended to be exhaustive. Preferred vectors for use according to the method of the present invention include any of those in the list consisting of the following: a fragment of a chromosome comprising, preferably containing a complete Ig locus (preferably a complete human Ig locus), a human artificial chromosome vector comprising, preferably containing complete Ig locus (preferably a complete human Ig locus) and a human artificial chromosome or YAC containing at least a portion of an Ig locus (preferably a human Ig locus).

Transfection of Donor Cells.

Suitable methods for the transfection of vectors referred to above into donor cells includes any one or more of those methods in the list consisting of the following: eletroporation, micro-injection, liposomal transfer, spheroplast transfection and viral transduction. Furthermore, whole chromosomes or chromosome fragments can be transfected into donor cells by cell fusion or microcell fusion. These methods will be familiar to those skilled in the art. Further, other such methods will be apparent to those in the art.

Spheroplast fusion as a method of transferring YACs into mammalian cells has proven to be a successful method for large YACs (Huxley and Gnirke, 1991). The yeast cells harbouring the YACs are prepared by enzymatic digestion of the cell wall to create spheroplasts. A proportion of the spheroplasts will carry the YAC DNA, with varying amounts of yeast DNA, which are fused to ES cells using PEG. The YAC typically consists of a compromised version of a human Ig loci fused with a selectable marker which facilitates the recovery of the appropriate ES colonies under drug selection. YACs encoding antibody loci can be efficiently transferred into ES cells using lipid transfection methods, (Lee, and Jaenisch, 1996) Moreover, YACs as large as 2.3 Mb have been transfected into human cells (Marschall P., et al. 1999). Typically the YAC DNA is purified by pulse field gel electrophoresis, combined with a low salt buffer to compact the DNA and coated with a positively charged polyamine based carrier that creates a fusogenic molecule to the negatively charged cell plasma membrane.

The method of choice for larger molecules of DNA such as human artificial chromosomes, chromosome fragments or whole chromosomes is cell fusion and preferably PEG mediated microcell fusion, (Doherty and Fisher, 2003). Whole cells carrying the human chromosome of choice can be fused to ES cells by electrofusion or PEG mediated fusion, detailed description of the techniques are included within Example 1. The use of whole chromosomes as the fusion partners produces hybrid ES cells with ‘extra’ chromosomes. The hybrid cell lines are aneuploid and consist of a chromosome content made up from the two fusing cells. Preferably the donating fusion partner, (with the human chromosome of interest), is chemically fractionated into ‘mini-cells’ known as microcells. Each microcell consists of 1 or more chromosomes enveloped within a cell membrane which is then fused with an ES cell. Appropriate selection of the fusion products enables the recovery of a single genetically marked human chromosome within an ES cell. The procedure is further enhanced by the exposure of the microcells to γ radiation prior to fusion. Exposure to γ-rays has the desired effect of fragmenting the chromosome. As a general rule the smaller the human chromosome fragment the more mitotically stable it will be on transfer into a mouse ES cell.

Viral transduction of mammalian cells can result in efficient transfer of exogenous DNA fragments, (White et al., 2002). Generally speaking the viral vectors available are not suitable for the transfection of ES cells with large human Ig encoding vectors due to the limited DNA carrying capacity of the infectious viral particles. EBV viral vectors can package 120 Kb amplicons into infectious particles. However, unpackaged vectors consisting of the EBV sequences, crucially the origin of replication (oriP) and the EBV nuclear antigen 1 (EBNA1) integrated into a typical bacterial plasmid backbone, can carry larger fragments of 660 Kb, (White et al., 2002). Such a vector could not accommodate an intact (>1 Mb) human Ig locus, but a shortened version of an Ig locus consisting of essential immunoglobulin coding sequences could be maintained and transferred into mouse ES cells using such an EBV based vector. YACs as small as 320 Kb and 340 Kb representing truncated versions of the light and heavy chain loci respectively, have been successfully used to produce fully humanised antibodies in mice, (Bruggermann 2001). EBV based vectors can carry compromised but functional versions of the Ig heavy and light chain loci that could be transfected into ES cells by stanadard lipofection or elctroporation based methods.

Alternatively, the introduction of chromosomes (preferably human chromosomes) or fragments thereof can be achieved by firstly sorting the desired chromosome or fragment by FACS analysis and then introducing the purified fragments by micro-injection, (Monard, 1998).

Manipulation of Donor Cells Prior to Transplantation.

One embodiment of the present invention makes use of wild type donor cells in respect of their immunoglobulin light and heavy genes. This embodiment of the method therefore creates various hybrid molecules between mouse and human heavy and light chains (partially human antibodies) as well as fully human antibodies.

In an alternative embodiment of the invention therefore the method of the invention makes use of donor cell lines with their endogenous Ig loci inactivated, such that they are non-functional for the expression of mouse antibodies. Thus the engineered pluripotent cells give rise to progeny exclusively expressing antibodies of human origin. This can be achieved by inactivating the endogenous mouse Ig loci by site-specific deletion. Cre recombinase sponsored recombination of specifically integrated loxP sites flanking the heavy and light chain gene loci is the method of choice. This deletion approach creates donor cells, which once transplanted into recipient immuno-compromised mice, are incapable of producing functional mouse antibodies. However, the necessary cellular machinery for rearranging the trans-loci to produce the full immune repertoire of antibody molecules remains intact. Therefore, if such inactivated donor cells are engineered to express human Ig loci and transplanted to a mouse host, then the colonising B cells are capable of exclusively expressing human antibodies upon immunisation.

Those skilled in the art will appreciate that any alteration to mouse genes must be homozygous to be effective. Introducing a single deletion into ES cells and using the pluripotent cells to make chimaeric mice by standard methods, which can then be bred to homozygozity, can achieve this. The homozygous mutant ES cells can then be re-derived from the mice. Alternatively, the ES cell line with a deletion of an Ig cluster at one allele can be subjected to high levels of the selectable drug to cause gene conversion and homozygozity.

The Functional Inactivation of Donor Cells Endogenous Ig Locus

The removal of an Ig heavy or Ig light chain can be achieved by a 2-step gene targeting strategy that flanks the locus with loxP sites using standard gene targeting techniques in donor cells, in particular ES cells. Subsequently the donor cells with the floxed Ig loci can then be exposed to the transient expression of the Cre recombinase enzyme. This enzyme causes the site specific excision of the intervening DNA and hence the removal of one allele of a Ig loci. The deletion is made homozygous by the creation and subsequent breeding of mice from the altered donor cells. The homozygous mice are then used to produce new donor cell lines with deleted Ig loci.

This strategy requires that the locus be targeted at the 5′ and 3′ flanks with different selectable markers such that a lox P site is placed at an appropriate distance from the first V gene sequence and the second lox P site at the most distal C gene segment.

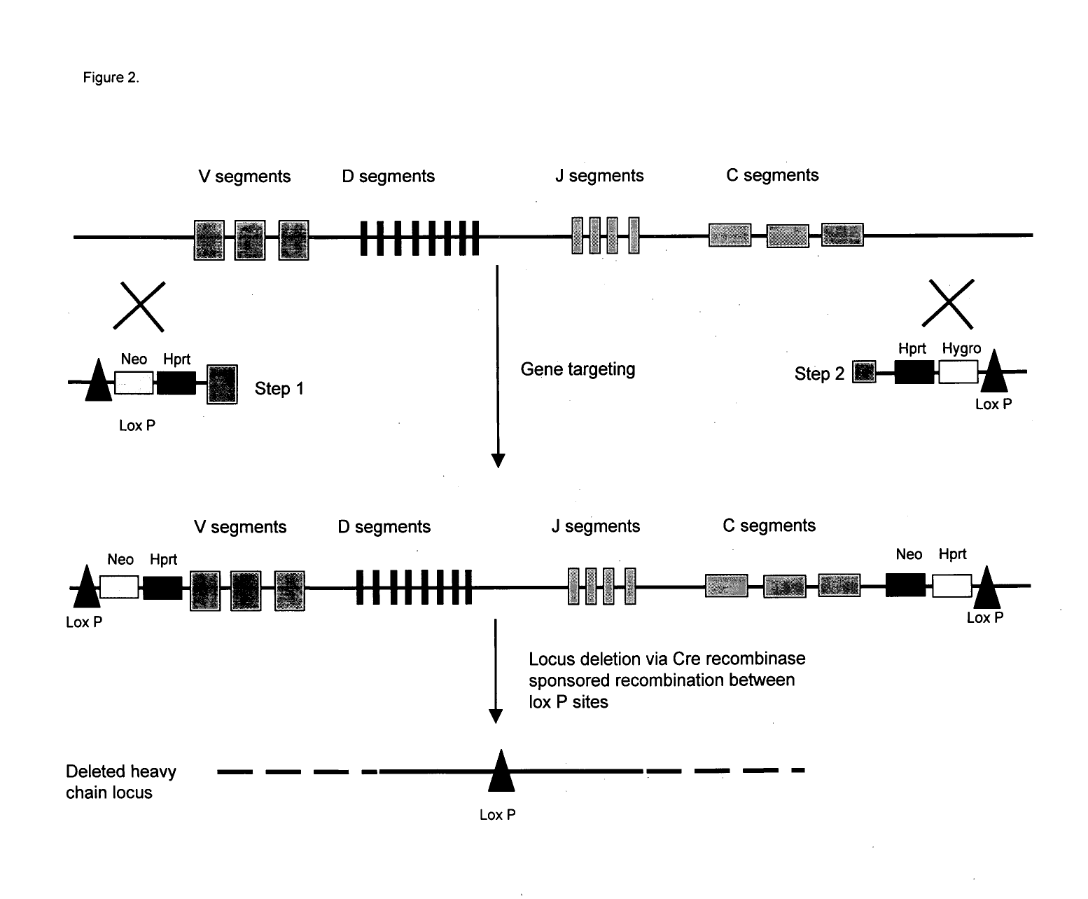

Step 1 requires the use of a targeting construct consisting of 6-10 kb of homology to the 5′ region interspersed by 2 selectable markers (for example Neomycin and HPRT) and a lox P site arranged in a suitable orientation, for example that outlined in FIG. 2. HM1 ES cells, which are HPRT deficient, are transfected with the linearised construct according to standard procedures. Donor cells that have integrated the first targeting construct will be resistant to the drug G418, and are therefore identified on the basis of their resistance. Resistant G418 clones that have targeted the correct gene locus distinguished from random integrants by Southern blot and/or PCR analysis.

The second targeting step requires a construct consisting of 6-10 kb of sequence homology to the 3′ end of the locus. The homologous gene sequences are engineered to introduce a LoxP site and the 2 selectable markers HPRT and Hygromycin as shown in FIGS. 2, 3 and 4. Correctly targeted cells from the first targeting step are re-transfected with the second construct. This produces doubly resistant Hygromycin and Neomycin cells. The desired (correctly targeted) doubly resistant cells are identified by Southern and/or PCR analysis. At this point LoxP sites flank the heavy chain locus. Transient expression of the cre recombinase enzyme catalyses site specific recombination between these two sites, and the deletion of the intervening DNA, which is the Ig gene locus. The deleted locus no longer has 2 HPRT genes integrated at its flanks, which allows 6-TG selection to kill those cells that have not deleted the locus as they still contain at least on functional copy of the HPRT gene and are therefore sensitive to 6-TG. Therefore resistant colonies may be the result of Cre sponsored deletion of the Ig locus. The locus deletion is verified by PCR and Southern blot analysis.

Details are Provided in Example 3 and FIGS. 2, 3 and 4.

According to the preferred method of this invention, the ES cells in which the endogenous Ig loci have been removed are the preferred cell type to which the human Immunoglobulin loci are added.

Differentation and Sorting of Donor Cells Prior to Transplantation into Recipient/Host Animals.

Transfected donor cells may be subjected to in vitro differentiation as hererin defined and cell enrichment using any suitable method for example FACS/MACS sorting and/or affinity based column purification. A detailed description of the methods for in vitro differentiation can be found in the methods of Example 4. Those skilled in the art will appreciate that this list is not intended to be exhaustive. Such techniques enrich for particular cell types prior to transplantation into a host animal. For example in the case where the donor cells are embryonic stem cells (ES cells) then such cells may be treated such that they differentiate into immature B-cells as herein defined including any one or more of those cells types in the group consisting of: haematopoietic stem cells (HSCs), pre-pro B-cells, pre-B cells and pro-B cells prior to transplantation. Such treatments ensure the transplanted cells specifically colonize the B-cells compartments of the donor animal. Further these differentiated cells may then be sorted and enriched for a particular cell type. Thus in this case, differentiated cells may be sorted and those cells which have differentiated into any one or more of those cell types listed above selected for transplantation into host animals according to the methods herein described.

In an alternative embodiment of the method of the invention, non-differentiated cells are transplanted into hosts, and some of those cells may correctly colonise the intended organ niche. Details of suitable techniques used in this aspect of the invention are described in Bjorklund et al. Embryonic stem cells develop into functional dopaminergic neurons after transplantation in a Parkinson rat model. Proc Natl Acad Sci USA. 2002

The problem with such an approach is that injected non-differentiated cells may aggregate forming clumps and potentially teratocarcinomas with only a small percentage of the cells correctly grafting and differentiating. The present inventors consider that these problems may be overcome using a variety of techniques:

(i) If the ES cells are delivered at a reduced cell density directly to the site of engraftment then the inventors consider that it is possible to have an efficient and functional route of cell delivery. Reducing the cell numbers injected into any one site may also avoid the problems of aggregation. Furthermore, injection of ES cells into the site of engraftment would alleviate the problems of homing to the correct site and provide the necessary environmental cues for correct differentiation.

(ii) Alternatively or in addition, the donor cells may be engineered to express a cell surface protein that improves the ability of the cell to associate with its target, again improving the low efficiency of this approach.

(iii) Alternatively or in addition to (i) and (ii) above, donor cells may be mixed, prior to injection, with a particular growth factor or cytokine that would trigger or enhance the differentation of donor cells, in particular the differentiation of ES cells to haematopoietic cells².

The Host Animal (for Generation of a Somatic Transgenic Animal).

According to the methods described herein, the animal may be any animal provided it is a mammal. Preferably, the animal is a non-human mammal such as a rodent and even more preferably a rat or mouse. In this regard, it is also preferred that the host animal is an “endogenous Ig locus knock out” animal as herein described.

By using host animals incapable of producing antibodies that include light chains or at the very least with only a reduced capacity to produce such antibodies, the method of the present invention enables the efficient production of large quantities of antibodies and antibody producing cells from a somatic transgenic animal according to the present invention upon challenge with a given antigen.

General Transgenic Techniques.

Methods for making germ-line transgenic animals are well known in the art and are described in Watson, J. D., et al., “The Introduction of Foreign Genes Into Mice,” in Recombinant DNA, 2d Ed., W. H. Freeman & Co., New York (1992), pp. 255-272; Gordon, J. W., Intl. Rev. Cytol. 115:171-229 (1989); Jaenisch, R., Science 240: 1468-1474 (1989); and Rossant, J., Neuron 2: 323-334 (1990). Techniques described therein such as techniques for the introduction of foreign DNA into ES cells are applicable to the method of the present invention. These documents are herein incorporated by reference.

Methods for the functional inactivation or suppression of endogenous Ig locus in host recipient animals are described above.

The Generation of Recipient Host Animals in Which the Endogenous Ig Locus has Been Suppressed and/or Inactivated.

Functional Disruption of Endogenous Immunoglobulin Loci.

Methods for the disruption of Endogenous Immunoglobulin loci are well known to those skilled in the art and are detailed in several patents including those in the following list: WO98/24884 in the name of Medarex, WO02/059154 and WO 942602 in the name of Abgenics. These documents are herein incorporated by reference.

One suitable way to generate a non-human somatic transgenic animal that is devoid of endogenous antibodies is by mutating the endogenous immunoglobulin loci. Using embryonic stem cell technology and homologous recombination, the endogenous immunoglobulin repertoire can be readily eliminated. The following describes the functional description of the mouse immunoglobulin loci.

The vectors and methods disclosed, however, can be readily adapted for use in other non-human animals.

Briefly, this technology involves the inactivation of a gene, by homologous recombination, in a pluripotent cell line that is capable of differentiating into germ cell tissue.

A DNA construct that contains an altered, copy of a mouse immunoglobulin gene is introduced into the nuclei of embryonic stem cells. In a portion of the cells, the introduced DNA recombines with the endogenous copy of the mouse gene, replacing it with the altered copy. Cells containing the newly engineered genetic lesion are injected into a host mouse embryo, which is reimplanted into a recipient female. Some of these embryos develop into chimeric mice that possess germ cells entirely derived from the mutant cell line. Therefore, by breeding the chimeric mice it is possible to obtain a new line of mice containing the introduced genetic lesion (reviewed by Capecchi (1989), Science, 244, 1288-1292). Such a technique is referred to as the ‘knocking out of a gene’. The host animals (in this case mice) created in this way are referred to as ‘endogenous Ig locus knock-out mice’. Thus according to the methods of the present invention, advantageously, ‘host animals’ in which the endogenous/native immune system has been suppressed and/or functionally inactivated refers to those host animals in which the endogenous Ig locus has been functionally inactivated using gene targeting techniques.

According to the methods of the invention, preferably endogenous Ig locus knock-out mice are either SCID mice (Dorshkind et al. 1984), RAG-1 (Mombaerts et al. 1992) or RAG-2 (Shinkai et al. 1992) knockout mice³.

Because the mouse X locus contributes to only 5% of the immunoglobulins, inactivation of the heavy chain and/or K-light chain loci is sufficient. There are three ways to disrupt each of these loci, deletion of the J region, deletion of the J-C intron enhancer, and disruption of constant region coding sequences by the introduction of a stop codon. The last option is the most straightforward, in terms of DNA construct design. Elimination of they gene disrupts B-cell maturation thereby preventing class switching to any of the functional heavy chain segments. The functional disruption of the mouse immunoglobulin loci is presented in the Examples.

Suppressing the Expression of Endogenous Ig Loci.

In addition to functional disruption of endogenous Ig loci, an alternative method for preventing the expression of an endogenous Ig locus is suppression. Suppression of endogenous Ig genes may be accomplished by the use of any one or more of the following techniques: with antisense RNA produced from one or more integrated transgenes, using small double stranded RNA molecules to cause RNA interference, by antisense oligonucleotides, by administration of antisera specific for one or more endogenous Ig chains and/or by the administration of antibodies specific for nucleic acid encoding the respective Ig locus. Advantageously, preventing the expression of an endogenous Ig locus involves using small double-stranded RNA molecules to cause interference⁴. Those skilled in the art will appreciate that this list is not intended to be exhaustive and will be aware of other suitable techniques.

Antisense Polynucleotides

Antisense RNA transgenes can be employed to partially or totally knock-out expression of specific genes (Pepin et al. (1991) Nature 355: 725; Helene., C. and Toulme, J. (1990) Biochimica Biophys. Acta 1049: 99; Stout, J. and Caskey, T. (1990) Somat. Cell Mol. Genet. 16: 369; Munir et al. (1990) Somat. Cell Mol. Genet. 16: 383, each of which is incorporated herein by reference) “Antisense polynucleotides” are polynucleotides that: (1) are complementary to all or part of a reference sequence, such as a sequence of an endogenous Ig CM or CL region, and (2) which specifically hybridize to a complementary target sequence, such as a chromosomal gene locus or a Ig mRNA. Such complementary antisense polynucleotides may include nucleotide substitutions, additions, deletions, or transpositions, so long as specific hybridization to the relevant target sequence is retained as a functional property of the polynucleotide. Complementary antisense polynucleotides include soluble antisense RNA or DNA oligonucleotides which can hybridize specifically to individual mRNA species and prevent transcription and/or RNA processing of the mRNA species and/or translation of the encoded polypeptide (Ching et al., Proc. Natl. Acad. Sci. U.S.A. 86:10006-10010 (1989); Broder et al., Ann. Int. Med. 113:604-618 (1990); Loreau et al., FEBS Letters 274:53-56 (1990); Holcenberg et al., WO91/11535; U.S. Ser. No. 07/530,165 (“New human CRIPTOgene”); WO91/09865; WO91/04753; WO90/13641; and EP 386563, each of which is incorporated herein by reference).

Small inferencing RNS molecules siRNA may in addition or as an alternative be used to suppress and or functionally inactivate the endogenous Ig locus. RNA interference refers to the process of sequence-specific post-transcriptional gene silencing in animals mediated by short interfering RNAs (siRNAs) (Fire et al., 1998, Nature, 391, 806). The process of post-transcriptional gene silencing is thought to be an evolutionarily-conserved cellular defense mechanism used to prevent the expression of foreign genes and is commonly shared by diverse flora and phyla (Fire et al., 1999, Trends Genet., 15, 358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response though a mechanism that has yet to be fully characterized.

RNAi has been studied in a variety of systems. Fire et al., 1998, Nature, 391, 806, were the first to observe RNAi in C. elegans. Wianny and Goetz, 1999, Nature Cell Biol., 2, 70, describe RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J, 20, 6877) has revealed certain requirements for siRNA length, structure, chemical composition, and sequence that are essential to mediate efficient RNAi activity. These studies have shown that 21-nucleotide siRNA duplexes are most active when containing 3′-terminal dinucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the guide sequence (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

Studies have shown that replacing the 3′-terminal nucleotide overhanging segments of a 21-mer siRNA duplex having two-nucleotide 3′-overhangs with deoxyribonucleotides does not have an adverse effect on RNAi activity. Replacing up to four nucleotides on each end of the siRNA with deoxyribonucleotides has been reported to be well tolerated, whereas complete substitution with deoxyribonucleotides results in no RNAi activity (Elbashir et al., 2001, EMBO J., 20, 6877). In addition, Elbashir et al., supra, also report that substitution of siRNA with 2′-O-methyl nucleotides completely abolishes RNAi activity. Li et al., International PCT Publication No. WO 00/44914, and Beach et al., International PCT Publication No. WO 01/68836 preliminarily suggest that siRNA may include modifications to either the phosphate-sugar backbone or the nucleoside to include at least one of a nitrogen or sulfur heteroatom, however, neither application postulates to what extent such modifications would be tolerated in siRNA molecules, nor provides any further guidance or examples of such modified siRNA. Kreutzer et al., Canadian Patent Application No. 2,359,180, also describe certain chemical modifications for use in dsRNA constructs in order to counteract activation of double-stranded RNA-dependent protein kinase PKR, specifically 2′-amino or 2′-O-methyl nucleotides, and nucleotides containing a 2′-O or 4′-C methylene bridge. However, Kreutzer et al similarly fails to provide examples or guidance as to what extent these modifications would be tolerated in siRNA molecules.

Parrish et al., 2000, Molecular Cell, 6, 1977-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that RNAs with two phosphorothioate modified bases also had substantial decreases in effectiveness as RNAi. Further, Parrish et al. reported that phosphorothioate modification of more than two residues greatly destabilized the RNAs in vitro such that interference activities could not be assayed. Id. at 1081.

The use of longer dsRNA has been described. For example, Beach et al., International PCT Publication No. WO 01/68836, describes specific methods for attenuating gene expression using endogenously-derived dsRNA. Tuschl et al., International PCT Publication No. WO 01/75164, describe a Drosophila in vitro RNAi system and the use of specific siRNA molecules for certain functional genomic and certain therapeutic applications; although Tuschl, 2001, Chem. Biochem., 2, 239-245, doubts that RNAi can be used to cure genetic diseases or viral infection due to the danger of activating interferon response. Li et al., International PCT Publication No. WO 00/44914, describe the use of specific dsRNAs for attenuating the expression of certain target genes. Zernicka-Goetz et al., International PCT Publication No. WO 01/36646, describe certain methods for inhibiting the expression of particular genes in mammalian cells using certain dsRNA molecules. Fire et al., International PCT Publication No. WO 99/32619, describe particular methods for introducing certain dsRNA molecules into cells for use in inhibiting gene expression. Plaetinck et al., International PCT Publication No. WO 00/01846, describe certain methods for identifying specific genes responsible for conferring a particular phenotype in a cell using specific dsRNA molecules. Mello et al., International PCT Publication No. WO 01/29058, describe the identification of specific genes involved in dsRNA-mediated RNAi. Deschamps Depaillette et al., International PCT Publication No. WO 99/07409, describe specific compositions consisting of particular dsRNA molecules combined with certain anti-viral agents. Waterhouse et al., International PCT Publication No. 99/53050, describe certain methods for decreasing the phenotypic expression of a nucleic acid in plant cells using certain dsRNAs. Driscoll et al., International PCT Publication No. WO 01/49844, describe specific DNA constructs for use in facilitating gene silencing in targeted organisms.

All of these documents are herein incorporated by reference.

Antiserum Suppression

Partial or complete suppression of endogenous Ig chain expression can be produced by injecting mice with antisera against one or more endogenous Ig chains (Weiss et al. (1984) Proc. Natl. Acad. Sci. (U.S.A.) 81 211, which is incorporated herein by reference). Antisera are selected so as to react specifically with one or more endogenous (e.g., murine) Ig chains but to have minimal or no cross-reactivity with heterologous Ig chains encoded by an Ig transgene of the invention. Thus, administration of selected antisera according to a schedule as typified by that of Weiss et al. op.cit. will suppress endogenous Ig chain expression but permits expression of heterologous Ig chain(s) encoded by a transgene of the present invention. Suitable antibody sources for antibody comprise:

(1) monoclonal antibodies, such as a monoclonal antibody that specifically binds to a murine, y, K, or X chains but does not react with the human immunoglobulin chain(s) encoded by a human Ig transgene of the invention;

(2) mixtures of such monoclonal antibodies, so that the mixture binds with multiple epitopes on a single species of endogenous Ig chain, with multiple endogenous Ig chains (e.g., murine y and murine y, or with multiple epitopes and multiple chains or endogenous immunoglobulins;

(3) polyclonal antiserum or mixtures thereof, typically such antiserum/antisera is monospecific for binding to a single species of endogenous Ig chain (e.g., murine, murine y, murine K, murine A) or to multiple species of endogenous Ig chain, and most preferably such antisera possesses negligible binding to human immunoglobulin chains encoded by a transgene of the invention; and/or

(4) a mixture of polyclonal antiserum and monoclonal antibodies binding to a single or multiple species of endogenous Ig chain, and most preferably possessing negligible binding to human immunoglobulin chains encoded by a transgene of the invention.

Such B-cells may be used to generate hybridomas by conventional cell fusion and screened. Antibody suppression can be used in combination with other endogenous Ig inactivation/suppression methods (e.g., JM knockout, CM knockout, D-region ablation, antisense suppression, compensated frameshift inactivation).

Complete Endogenous Ig Locus Inactivation

In a preferred embodiment of the invention, the entire endogenous heavy and light chain loci are inactivated by any of various methods, including but not limited to the following: (1) functionally disrupting and/or deleting by homologous recombination at least one and preferably all of the endogenous heavy chain constant region genes, (2) mutating at least one and preferably all of the endogenous constant region gene sequences to encode a termination codon (or frameshift) and other methods and strategies apparent to those of skill in the art. Deletion of a substantial portion or all of the heavy chain constant region genes and/or D-region genes may be accomplished by various methods, including sequential deletion by homologous recombination using targeting vectors, especially based on the Cre/LoxP system and the like.

Various combinations of the inactivation and suppression strategies may be used to effect essentially total suppression of endogenous (e.g., murine) Ig chain expression.

Expressing One or More Heterologous Immunoglobulin Molecules From One or More Cells of Those Host Animals According to Step (a)

Upon antigen challenge (via immunization) of a somatic-transgenic animal obtained according to the method of the invention, the transgenic animal generates a polyclonal anti-sera comprising human immunglobulin molecules of interest. Monoclonal antibody producing hybridomas may be generated from antibody producing B cells derived from these immunized animals using techniques known to those skilled in the art.

Generation of Hybridomas and Human Monoclonal Antibodies Using Somatic-Transgenic Animal Derived B Cells

Recombinant DNA technology may be used to produce human antibodies according to the present invention using an established procedure, in bacterial or preferably mammalian cell culture. The selected cell culture system preferably secretes the human antibody product.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure human immunoglobulin preparations and allows scale-up to give large amounts of the desired human immunoglobulins. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired human immunoglobulins can also be obtained by multiplying mammalian cells in vivo. For this purpose, hybridoma cells producing the desired human immunoglobulins are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the immunoglobulins are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by immunofluorescent staining of cells expressing the desired target by immunoblotting, by an enzyme immunoassay, e.g. a sandwich assay or a dot-assay, or a radioimmunoassay.

For isolation of the human antibodies, those present in the culture supernatants or in the ascitic fluid may be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with the target molecule or with Protein-A.

A Preferred Protocol for the Preparation of Human Monoclonal Antibodies According to the Invention.

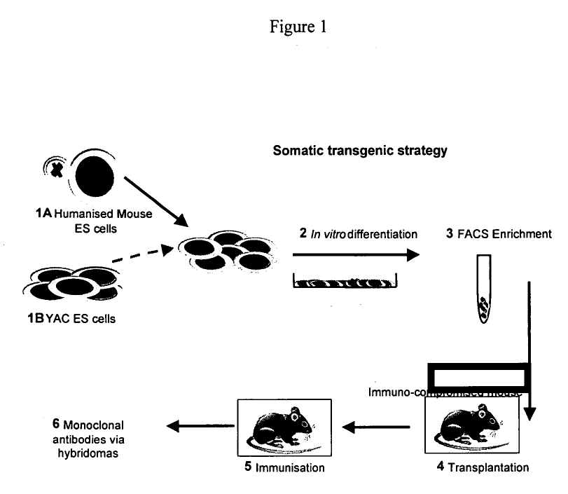

The present invention describes a novel method for the production of fully or partially human monoclonal antibodies from somatic transgenic mice. An example of the application of this new technology is outlined in FIG. 1:

(1) The production of pleuripotent cells or ES-like cells carrying the immunoglobulin genes of interest.

(2) Methods for the detection of human chromosomes 22, 14 and 2 or fragments thereof in those pleuripotent cells or ES-like cells.

(3) Methods for the differentiation of the pleuripotent cells or ES-like cells to haematopoietic stem cells (HSCs).

(4) Methods for pleuripotent cells or ES-like cell differentiation and enrichment for HSC/pro-B cells.

(5) A method for the transplantation of the Ig containing HSCs into mouse.

(6) Analysis of Ig gene expression before and after immunisation.

(7) Establishment of hybridomas expressing monoclonal antibodies.

This invention requires that pluripotent cells (in this example mouse embryonic stem cells) be modified to carry the immunoglobulin gene clusters. Here the inventors describe the introduction of the gene clusters by (i) chromosome fragments mediated by microcell fusion (ii) whole chromosomes mediated by cell fusion or microcell fusion (iii) YAC vectors containing portions of the Ig gene clusters. For the transfer of chromosome fragments or whole chromosomes to ES cell lines the inventors principally use mouse monohybrid lines containing the human chromosomes of interest (2, 14 or 22). The human chromosomes exist as intact whole chromosomes or rearranged versions (of), with the gene clusters of interest preserved in the majority of the cells. Individual human chromosomes are marked with selectable markers such as hygromycin, neomycin, blasticidin or in some cases a HPRT mini-gene which can be selected for in HAT media and against with 6-TG The selectable marker facilitates the recovery of ES cell lines, which have successfully incorporated the desired chromosome or fragment. For the individual transfer of chromosomes, the preferred protocol is that of microcell PEG mediated fusion of microcells. Prior to fusion the microcells are exposed to ionising radiation to fragment the chromosomes and then fused to the ES cells using PEG or electrofusion. Spheroplast fusion is used to transfer YACs into the ES cells.

PCR analysis for presence of DNA markers specific for the chromosome of interest assesses the integrity of the transferred chromosomes. Those clones with a PCR profile indicative of an intact Ig locus are furthered processed by FISH analysis. Those lines containing 1 or more copies of the desired chromosome in the majority of the cells are taken forward for further manipulation. The addition of another chromosome is achieved in a similar manner as the first. The major difference being that the second chromosome(s) is marked with a different selectable marker to the first. A third chromosome is added with similar consideration to marker usage. The chromosome content of the humanised ES cell line is verified by FISH and PCR prior to its usage in transplantation.

The present inventors have described the use of whole chromosomes, chromosome fragments or YACs containing portions of human chromosomes 14, 22 and 2 encoding for the immunoglobulin light and heavy chains for the expression of these genes within mice. The method described herein can also be performed using artificial human chromosomes for example; chromosome 22 and 14 can be engineered to contain loxP sites, these sites can recombine in the presence of Cre recombinase to generate a mini chromosome via a translocation between the two 2 chromosomes. The resulting mini chromosome could consist of the human heavy and light chain genes for chromosomes 14 and 22. The obvious advantage is that only one exogenous DNA element instead of 2 chromosome fragments has to be maintained within the ES cell line for successful human antibody expression.

The humanised pluripotent cells encode the necessary genetic material to express human antibodies in vivo. Others have accomplished this by using the totipotent cells to make chimaeric mice by the traditional method of blastocyst injection and subsequent necessary extensive breeding. The somatic transgenic approach shortcuts this necessity by directly transplanting the humanised ES cells into immuno-compromised donor mice. However, generally, the pluripotent stem cells must be committed to differentiating along the B cell lineage for efficient engraftment and colonisation and subsequent expression of human antibodies from the donor cells in the recipient/host animals. This invention therefore, in addition, details the process of HSC derivation from the humanised ES cells and the methods of transplantation to permit the cells to be incorporated into the proper cellular environment for their efficient engraftment and proliferation.

The pluripotent ES cells are committed to their developmental path using specialised in vitro culture methods. The preferred method requires the cells be cultured in a specific cocktail of cytokines with methylcellulose culture media as detailed in Example 2. This culture regime produces a heterogeneous population of cells in which exist HSCs and/pr stem-like cells which can colonise the B cell compartment on engraftment. The HSCs are purified from the mixed population of cells by FACS or MACS on the basis of their cell surface markers. The HSC or haemangioblast is poorly defined in this respect although sorting for expression of markers such as Flk1⁺, CD45⁺ and c-kit⁺ can enrich for the desired reconstituting B cells.

The enriched population cells are then transplanted into an immuno-compromised animal. Suitable immuno-compromised mice includes lethally irradiated mice as the host animals. Other suitable immuno-compromised host animals include SCID or Rag1^{-/-} mutant mice for example. The added advantage of such a modification is that the deficient endogenous B cells are not able to elicit an immune response, thus simplifying the process of isolating a human monoclonal antibody.

The efficiency with which the HSC derived cells engraft is highly dependent upon the route taken for transplantation. This invention described herein favours the intra bone marrow (IBM) method of injection for high levels of chimaerism within the bone marrow. Injection of purified cells directly into the bone marrow alleviates the need for these cells to home, via the circulatory system, to their desired niche. Direct IBM injection facilitates the proliferation and spread of HSC cells within the bone marrow for the reconstitution of the immune system of the immuno-compromised mice.

Immunisation of the somatic transgenic mice generated using the method of the invention stimulates the production of antibodies from those mice. Splenocytes of a B-cell lineage expressing human antibodies are isolated and fused with myeloma cells to generate hybridomas for the production of monoclonal human antibodies by standard methods.

Uses of Antibodies Obtained Using the Method of the Invention

Antibody molecules according to the present invention, preferably scFv molecules may be employed in in vivo therapeutic and prophylactic applications, in vitro and in vivo diagnostic applications, in vitro assay and reagent applications, in functional genomics applications and the like.

Therapeutic and prophylactic uses of antibodies and compositions according to the invention involve the administration of the above to a host mammal, such as a human. Preferably they involve the administration to the intracellular environment of a mammal.

Substantially pure antibodies of at least 90 to 95% homogeneity are preferred for administration to a mammal, and 98 to 99% or more homogeneity is most preferred for pharmaceutical uses, especially when the mammal is a human. Once purified, partially or to homogeneity as desired, the antibody molecules may be used diagnostically or therapeutically (including extracorporeally) or in developing and performing assay procedures using methods known to those skilled in the art.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the selected antibodies of the present invention in protecting against or treating disease are available. Suitable models will be known to those skilled in the art.

Generally, the selected antibodies of the present invention will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The selected antibodies of the present invention may be used as separately administered compositions or in conjunction with other agents. These can include various immunotherapeutic drugs, such as cylcosporine, methotrexate, adriamycin or cisplatinum, and immunotoxint. Pharmaceutical compositions can include “cocktails” of various cytotoxic or other agents in conjunction with antibodies of the present invention or even combinations of the antibodies, according to the present invention.

The route of administration of pharmaceutical compositions according to the invention may be any of those commonly known to those of ordinary skill in the art. For therapy, including without limitation immunotherapy, the selected antibodies of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician.

The selected antibodies of the present invention can be lyophilised for storage and reconstituted in a suitable carrier prior to use. Known lyophilisation and reconstitution techniques can be employed. It will be appreciated by those skilled in the art that lyophilisation and reconstitution can lead to varying degrees of functional activity loss and that use levels may have to be adjusted upward to compensate.

The compositions containing the present selected antibodies of the present invention or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected immunoglobulin per kilogram of body weight, with doses of 0.05 to 2.0 mg/kg/dose being more commonly used. For prophylactic applications, compositions containing the present selected immunoglobulin molecules or cocktails thereof may also be administered in similar or slightly lower dosages.

A composition containing one or more selected antibody molecules according to the present invention may be utilised in prophylactic and therapeutic settings to aid in the alteration, inactivation, killing or removal of a select target cell population in a mammal. In addition, the selected repertoires of polypeptides described herein may be used extracorporeally or in vitro selectively to kill, deplete or otherwise effectively remove a target cell population from a heterogeneous collection of cells. Blood from a mammal may be combined extracorporeally with the selected antibodies, cell-surface receptors or binding proteins thereof whereby the undesired cells are killed or otherwise removed from the blood for return to the mammal in accordance with standard techniques.

The invention will now be described by way of the following examples which should in no way be considered limiting of the invention.

### EXAMPLE 1

Methods for the Production of Embryonic Stem (ES) Cells Carrying the Human Immunoglobulin Genes.

Transfer of human chromosomes or fragments thereof into mouse embryonic stem cells was mediated by PEG sponsored micro-cell fusion. Micro-cells were prepared from human chromosome donor cells. These donor cells include primary human fibroblasts, telomerase immortalised human cells, SV40 transformed human cells, mouse ES cells, mouse 3T3 cells and mouse A9 cells containing the relevant human chromosome, (2, 14 or 22).

Mouse embryonic stem cells were grown in ES medium: Glasgow's Minimum Essential Medium (GMEM; Sigma) supplemented with 5% newborn calf serum and 10% fetal calf serum (Globepharm), 0.1 mM non-essential amino acids (Life technologies), 0.1 mM β-mercaptoethanol, 1 mM sodium pyruvate (Life technologies), 2 mM L-glutamine (Life technologies) and 1000 U/ml recombinant murine leukaemia inhibitory factor (LIF; Life technologies). The donor cells were grown in GMEM or DMEM (Gibco-BRL) supplemented with 10% foetal calf serum. The donor cells were grown to 70-80% confluence and treated with 0.05-0.15 μg/ml colcemid for 24 hours. The cells were harvested and resuspended in pre-warmed 1:1 mix of Percoll (Pharmacia) and serum-free DMEM containing 10 μg/ml cytochalasin B. The suspension was then centrifuged in a Beckman JA20 rotor at 19000 rpm at 37° C. for 75 min. Micro-cells were collected and washed twice in serum-free DMEM.

At this point the microcells can either be directly fused to the ES cells or the optional step of exposure to ionising radiation (1.2 Gy/min for 50 minutes) to fragment the chromosomes prior to fusion. The microcells (irradiated or not) were mixed with 1 to 5×10⁸ ES cells and fused using 1 to 2 ml of 50% PEG (polyethylene glycol) at room temperature for 1 minute. 10 ml serum free GMEM was added and the fusion was left at room temperature for 30 min. After two washes in serum free GMEM, the fused cells, were plated onto 5 to 10 pre-gelatinised 15 cm plates containing 30 ml ES maintainance media. The cells were cultured at 37° C. for 24 to 48 hours without selection. Selection was applied with the appropriate antibiotic, Hygromycin B (Sigma) at 100 to 250 μg/ml or G418 (Geneticin, Sigma) at 150 to 300 μg/ml. Media was changed frequently, once every 2 days or when required. Drug resistant colonies were picked from 2 to 3 weeks after selection was applied. The colonies were expanded, harvested for DNA analysis frozen down and processed for FISH analysis when required.

The preferred method of the invention requires the use of ES cells derived from endogenous Ig locus knock-out mice as detailed in Example 3. Other mouse ES cells used were either the HPRT deficient HM-1 ES line (Thompson et al. 1989) derived from the 129 mouse strain or the ED1 line derived from the F1 cross of C57/Bl and 129sv. The A9(Hygro 22) and A9(Hygro 14) cells are maintained at a concentration of Hygromycin B of 1 mg/ml to preserve the unstable human chromosomes (Tanabe et al. 2000). Mouse A9 cells containing human chromosome 22, 14 or 2 marked with a selectable neomycin gene were also used, (Coriell Cell Repositories, Coriell Institute for Medical Research, New Jersey, USA).

Whole cell to cell fusions were also performed between human chromosome/mini-chromosome containing cells and ES cells. Typically, 1×10⁸ ES cells were fused with 1×10⁸ cells mouse A9 monohybrid cell line for chromosome 22 or 14. All cells are harvested and washed 3× with 1×PBS. The ES cells and their fusion partners are mixed at ratios from 1:1 to 1:5. The mixed cells were spun down (100 g for 5 min) and re-suspended in 400 μl of 0.3M mannitol. Prior to electrofusion, the cell mix was spun down in a cuvette (gap distance of 0.2 cm) at 200 g inside 50 ml falcon tubes insulated with tissue paper. The cells were electrofused under various conditions. A pulse of 300V with a capacitance of 25 μF was found to be optimum when using a Biorad genepulser with 0.2 cm cuvettes. The fused cells in the cuvette were allowed to sit at room temperature for 15-20 min prior to plating. A single fusion experiment was plated onto 10×15 cm round plates with ES media. After 24 hours selection was applied, where appropriate either HAT alone or HAT with G418 @ 250 μg/ml or Hygromycin or Blasticidin was applied at the appropriate concentration

The human Immunoglobulin loci, or fragments of, were also introduced into ES cells using YAC vectors. 50 ml of selected media was inoculated with the appropriate yeast strain. Cells were shaken at 30° C. for approximately 2 days (OD₆₆₀=2.0). 1000D units of cells were collected by centrifugation @ 1000 g for 5 minutes. The cell pellet was washed in 25 ml of sterile water, centrifuged and re-suspend in 25 ml of 1 M sorbitol The cells were centrifuged and re-suspend in 15 ml of SCE with 75 μl of 2 M DTT. 500 μl of SCE containing 10-200 mg of yeast lytic enzyme (YLE) was added. The OD was empirically increased by −10-20% in <40 minutes. The suspension was incubated at 30° C. with occasional shaking. At this point the OD₆₆₀ was 10-20% of the initial OD₆₆₀ reading. Cells were centrifuged at 400 g for 5 minutes and then washed in 15 ml of STC×2. Centrifuged and re-suspended in 2 ml of STC. Adherent mammalian cells that were growing at log phase were collected. Washed twice in serum-free tissue culture media and re-suspended at a concentration of 2×10⁶ cells/ml. 2×10⁶ cells were added to a 15 ml conical polystyrene tube. Cells were centrifuged and 10D unit of protoplasts (1×10⁸ spheroplasts) were layered on top of the cells. The yeast protoplasts were centrifuged onto the cells and STC media removed. 50 ml of serum-free media was added and the pellet gentlt resuspended. After the cells are re-suspended, 500 ml of a 50% sterile solution of polyethylene glycol 1500 (PEG 1500, Boehringer-Mannheim #783641) and 3 ml of 1 M CaCl₂ were added. Incubated at room temperature for 2 minutes and then 5 ml of serum-free media was added and incubated at room. Cells were collected by centrifugation, re-suspend in ES media and plated onto four 10-cm tissue culture dishes. After 24 hours incubation cells were fed and at approximately 48 h after the fusion, selection was added. Resistant clones appeared between 14 and 21 days

Alternatively, YAC DNA was transfected directly into the ES cells by lipofection, see Lee and Jaenisch (1996), A method for high efficiency YAC lipofection into murine embryonic stem cells.

### EXAMPLE 2

Detection of Human Chromosomes 22, 14 and 2 or Fragments Thereof in ES Cells

Detection of human chromosome or fragments thereof in ES cell lines was initially performed by PCR analysis using human specific PCR primers. PCR positive lines were then verified.

Detection of chromosomes (2, 14 and 22) and fragments of, were initially detected using PCR for DNA markers specific for the respective chromosomes. Genomic DNA was prepared from the ES hybrid lines by the following method a method similar to the ‘Nucleon I’ DNA extraction kit method of Scotlab. Briefly, the ES cell clones were expanded in a 24 well plate. Cells were then trypsinised with 100 μl Trypsin solution for 3 minutes then media was added to stop reaction and the cells were pelleted at 2000 rpm. for 5 min. The cell pellets were re-suspended by gentle vortexing in 340 μl of reagent B (400 mM Tris-HCl pH to 8.0 using 2 M NaOH. 60 mM EDTA. 150 mM NaCl. 1% SDS). 2 μl of 10 mg/ml RNase A was added and incubated at 37° C. for 15-20 min. A 100 μl of 5 M sodium perchlorate was added to each tube and incubated at 37° C. for 20 min with occasional inversion. Samples were then incubated for a further 20 min at 65° C. with occasional inversion. 580 μl of chloroform chilled on dry ice, was added and samples rotary mixed at room temperature for 20 min. Centrifugation at 12000 rpm. for 20 min was followed by the careful removal of the supernatant to a fresh microfuge tube. The above chloroform step was repeated. 2 volumes of 100% ethanol, (750 μl), were added to precipitate the DNA. Samples were subsequently centrifuged at 12000 rpm. for 10 min. Pellets were washed with and 500 μl of 70% ethanol and allowed to dry at room temperature. The DNA pellet was resuspended in 60 μl distilled water.

All PCR genotyping was performed on the DYAD Peltier thermal cycler in 50 μl reaction mixtures. When using microsatellite markers, template genomic DNA of 0.2-1 μg was used with primers at a final concentration of 0.2 μm. Cycle conditions were; an initial 10 min cycle of 94° C., followed by 40 cycles of 94° C. for 20 seconds, 55° C. for 60 seconds and 72° C. for 30 seconds. Reactions were resolved on 4% Metaphor agarose from BMA. When PCR primers were designed to amplify a larger target molecule (500 bp or greater) then only 30 cycles of amplification were used and the reactions resolved on a 2% agarose gel.

Some PCR Primer Pairs for Markers on Chromosome 22

IGLC (Ig lambda constant region, Tomizuka et al., 1997).

5′-GGAGACCACCAAACCCTCCAAA-3′ and 5′-GAGAGTTGCAGAAGGGGTGACT-3′ product size 481 bp.

VλIII (Williams and Winter, 1993). 5′-GGAATTCAAGAACTTGGACCACTGT-3′ and 5′-CCCCCAAGCTTCTTTCCAGGCTCCTGGGC-3′ product size 511 bp

ARSA:

5′-GAGAGACACAGGCACGTAGAAG-3′ and 5′-GGCTATGGGGACCTGGGCTG-3′ product size 110 bp.

DNA primers for polymorphic markers D22S257 and D22S264

Some PCR Primer Sets for Chromosome 14

DNA primers for polymorphic markers DS412, D4S418, D4S395, D4S413 and D4S426 TCRA:

5′-AAG TTC CTG TGA TGT CAA GC-3′ and 5′-TCA TGA GCA GAT TAA ACC CG-3′

IGM: 5′-GCA TCC TGA CCG TGT CCG AA-3′ and 5′-GGG TCA GTA CCA GTG CCA G-3′

Some PCR Primer Sets for Chromosome 2

DNA primers for polymorphic markers D2S207, D2S177, D2S156 and D2S159

Fluorescence in Situ Hybridisation

ES lines which were PCR positive for human chromosome or fragments thereof were verified by FISH. The human specific DNA probes used were labelled by Nick Translation: 1 μg of DNA was re-suspended in a total of 25 μl of dH₂O 2.5 μl nick translation buffer (10×: 10 ml 1 M Tris pH7.4, 2 ml 1M MgSO₄, 20 μl 1M DTT, 1 ml 10 mg/ml BSA, 6.98 ml ddH₂O) and 1.9 μl dNTP's (0.5 mM A, C, G+0.25 mM T) and 0.7 μl biotin-16-dUTP or digoxigenin-11-dUTP (1 nm/μl) and 1 μl DNAse I (1 μg/ml) and 1 μl DNA polymerase I (10 Units/μl). Reaction was incubated at 16° C. for 35 min and stopped with 1 μl (1/10 vol.) 0.5 M EDTA @ 65° C. for 10 min. 2 ml of the labelled probe was run on 0.8% agarose gel to check size, (optimum size <500 bp).

Metaphase Slide Preparation of ES Cell Lines:

ES cells were split such that they were 70-80% confluent on morning of prep. They were fed 3 hours prior to colcemid treatment of a final concentration of 0.1 ug/ml colcemid for 1 hour prior to harvest. Cells were trypsinised in the usual manner, centrifuged at 1000 rpm for 5 minutes and supernatant removed. A total of 8 mls of fresh ice-cold hypotonic solution was added drop-wise, (Hypotonic solution is 0.075 M KCl), and incubated at room temp for 10 minutes. Cells were spun for 5 min at, hypotonic solution removed and replaced with 8 ml of ice-cold FIX solution, (FIX solution was a 3:1 ratio of Methanol to Acetic acid). After 10 min at room temperature 5 mins, cells were spun at 1000 rpm for 5 min, the supernatant, was removed and the FIX treatment repeated twice and finally the cells were re-suspended with 0.5 ml of cold FIX. Clean slides stored in ice cold 70% ethanol, were washed in cold tap water immediately before use. The cells were dropped from about 45 cm onto slides at 45° angle and dried at room temp. the slides were then dehydrated in an ethanol series of 70%, 90% and 100% for 5 min each then air dried and incubated in an oven at 65° C. for about 1 hour.

Hybridisation of Probe to Slides

Slides were immersed in a denaturing solution of 70% formamide/2×SSC at 65° C. for 1 min and immediately immersed in cold 70% ethanol for 5 min and the ethanol series repeated of 70%, 90% and 100%—5 min each and then air dried. Meanwhile the DNA probe was prepared by mixing 1 μl of probe mix with 0.5 μl salmon sperm DNA (10 mg/ml) and 14 μl hybridization mix (Hybridisation mix: 25 ml deionised formamide, 10 ml 50% dextran sulphate 5 ml 20×SSC (filtered) 4 ml 0.5 M sodium phosphate pH7 (2.3 ml 0.5 M Na2HPO4+1.7 ml 0.5 M NaH2PO4) 1 ml 50× Denhardt's solution). Incubated at 65° C. for 10 min and left at 37° C. for 30 min. The probe was then added to slide and sealed with a coverslip and incubated overnight at 37° C. in a moist chamber.

Post-Hybridization Detection

Coverslips were removed by rinsing with 2×SSC. Excess probe was removed by washing twice with 50% formamide and 2×SSC for 5 min at 42° C. Then twice with 0.1×SSC @ 42° C. for 5 min and subsequently with 4×SSC/0.1% Tween @ room temperature for 5 min. Then 100 μl of blocking buffer (4×SSC; 0.1% Tween; 3% BSA) was added under parafilm to the slide and incubated @ 37° C. in moist chamber for 20 min. Subsequently, a 100 μl of FITC solution (1:200 dilution in blocking buffer) was added to the slide, under parafilm, 37° C. in moist chamber for 30 min. The slide was then washed 3 times with 4×SSC/0.1% Tween @ room temp and the slide mounted with Vectashield+DAPI. For double labelling one probe was labelled with dig-11-dUTP and one with biotin-16-dUTP. Detection was with anti-DIG-FITC (Boehringer) and with streptavidin-Cy3 (Amersham). For each slide, 6 μl of anti-DIG-FITC and 0.3 μl of streptavidin-Cy3 to 120 μl blocking buffer (4×SSC; 0.1% Tween; 3% BSA) was added. The slide was then incubated with 100 μl of this. The DAPI mounting medium comes from Vector labs (Vectashield+DAPI).

### EXAMPLE 3

Methods for the Construction of Mouse ES Cells with Deleted Ig Loci.

Construction of Targeting Vectors for Inactivating the Ig Heavy Locus

The inactivation required 2 targeting vectors to be built. The targeting strategy along with the relative order of the 2 cassettes are presented in FIG. 2. The first construct integrated at the 5′ end of the first variable gene segment. The vector consisted of 6 to 10 kb of homology to the intronic sequence 5′ of the first variable gene sequence together with the first variable gene sequence. The required genomic DNA fragments for both targeting constructs were isolated by long range PCR from mouse 129sv genomic DNA according to the manufacturers instructions of the EXPAND PCR system (Boehringer Mannheim). The PCR derived fragments were cloned into the pGemT-Easy vector system (Promega). The selectable markers neomycin and a HPRT mini-gene are included along with a loxP site within the mouse sequence of the targeting vector. The flanking homology was not less than 1 kb on any one side of the construct. The second targeting construct required 6 to 10 kb of homology to the 3′ intronic sequence flanking and including the most distal constant gene sequences. A hygromycin and HPRT mini-gene were included as selectable markers along with a loxP site and were placed within the arms of homology in the order indicated in FIG. 2. The constructs were targeted to the Ig Heavy locus sequentially. Clones that have been correctly targeted at both ends of the Ig locus are transiently exposed to Cre recombinase. Deletion of the sequence intervening the loxP sites produces ES cell lines that are hemizygous for the desired deletion ie lacking one allele of the Heavy locus. This cell line was used to make a homozygous KO mouse line for the Ig Lambda locus. Details of the methods for DNA ligations, vector construction ES cell transfections, ES cell selections, screening of recombinant and deleted ES clones, making KO mice and isolation of ES lines are detailed in the following sections.

Construction of Targeting Vectors for Inactivating the Ig Kappa Locus

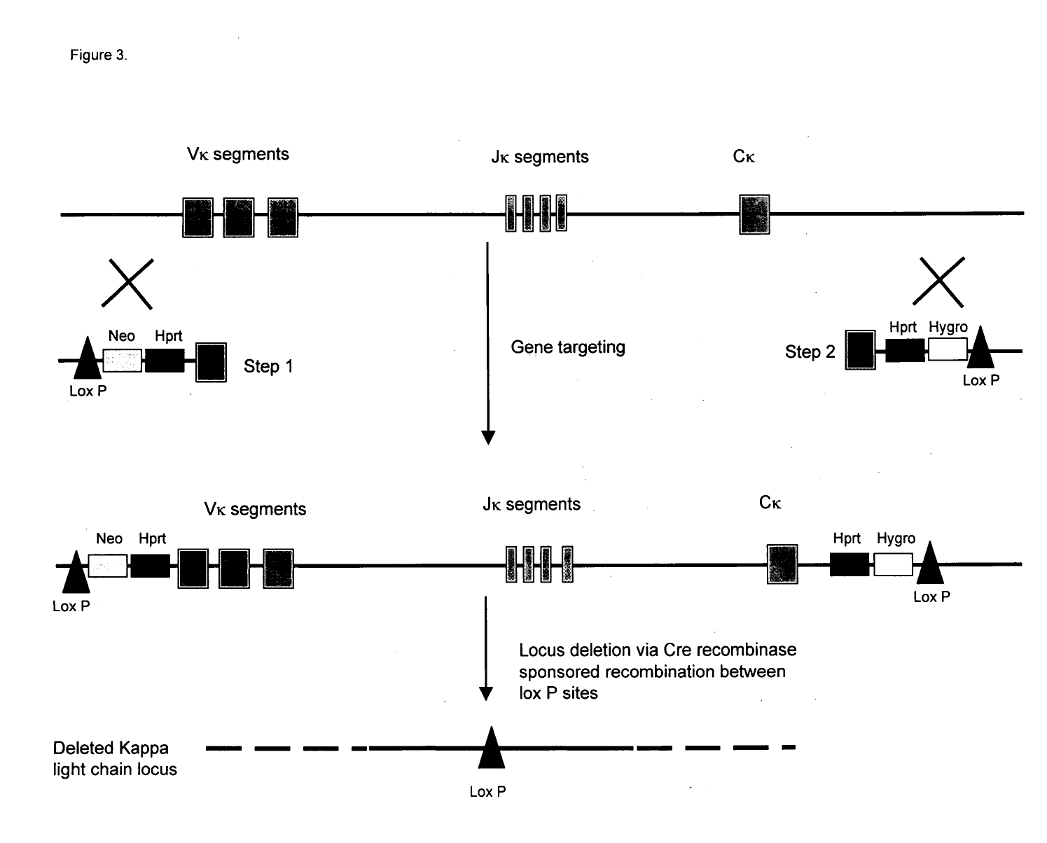

The inactivation required 2 targeting vectors to be built. The targeting strategy along with the relative order of the 2 cassettes are presented in FIG. 3 The first construct integrated at the 5′ end of the first variable gene segment. The vector consisted of 6 to 10 kb of homology to the intronic sequence 5′ of the first variable gene sequence together with the first variable gene sequence. The required genomic DNA fragments for both targeting constructs were isolated by long range PCR from mouse 129sv genomic DNA according to the manufacturers instructions of the EXPAND PCR system (Boehringer Mannheim). The PCR derived fragments were cloned into the pGemT-Easy vector system (Promega). The selectable markers neomycin and a HPRT mini-gene are included along with a loxP site within the mouse sequence of the targeting vector. The flanking homology was not less than 1 kb on any one side of the construct. The second targeting construct required 6 to 10 kb of homology to the 3′ intronic sequence flanking and including the most distal constant gene sequences. A hygromycin and HPRT mini-gene were included as selectable markers along with a loxP site and were placed within the arms of homology in the order indicated, FIG. 3. The constructs were targeted to the Ig Heavy locus sequentially. Clones that have been correctly targeted at both ends of the Ig locus are transiently exposed to Cre recombinase. Deletion of the sequence intervening the loxP sites produces ES cell lines that are hemizygous for the desired deletion ie lacking one allele of the lambda locus. This cell line was used to make a homozygous KO mouse line for the Ig Kappa locus. Details of the methods for DNA ligations, vector construction ES cell transfections, ES cell selections, screening of recombinant and deleted ES clones, making KO mice and isolation of ES lines are detailed in the following sections.

Construction of Targeting Vectors for Inactivating the Ig Lambda Locus

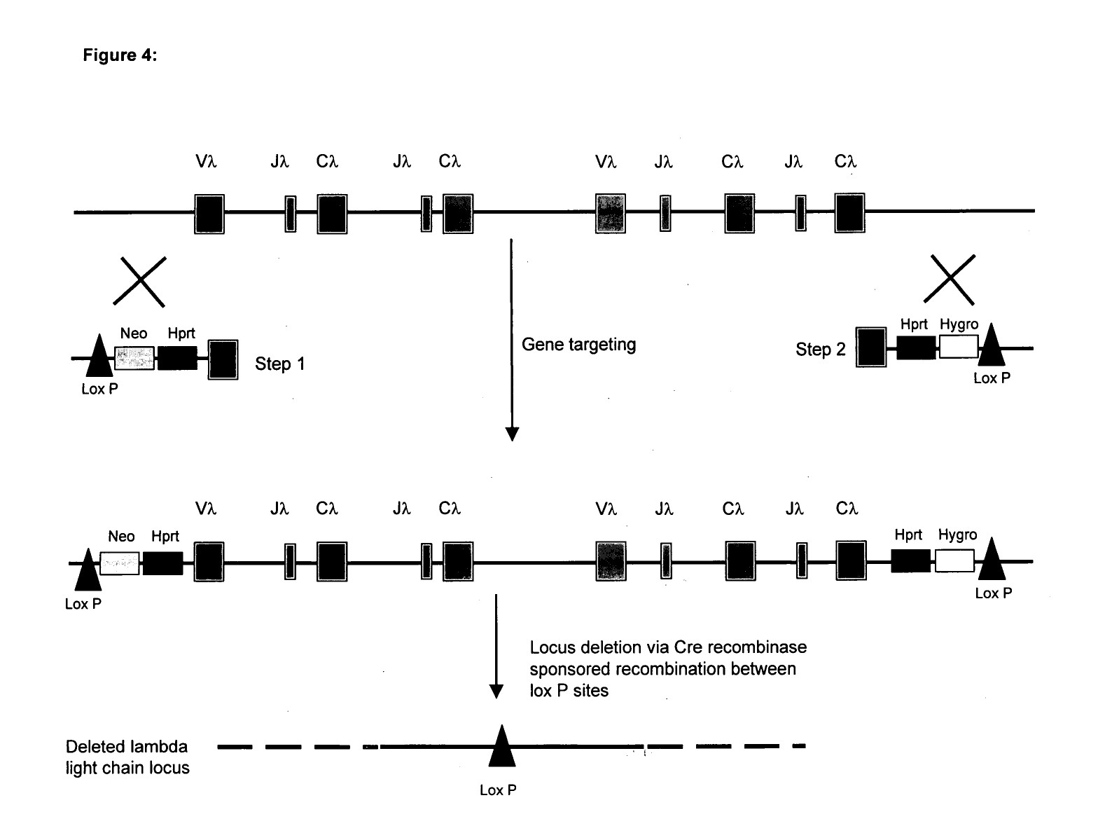

The inactivation required 2 targeting vectors to be built. The targeting strategy along with the relative order of the 2 cassettes are presented in FIG. 4. The first construct integrated at the 5′ end of the first variable gene segment. The vector consisted of 6 to 10 kb of homology to the intronic sequence 5′ of the first variable gene sequence together with the first variable gene sequence. The required genomic DNA fragments for both targeting constructs were isolated by long range PCR from mouse 129sv genomic DNA according to the manufacturers instructions of the EXPAND PCR system (Boehringer Mannheim). The PCR derived fragments were cloned into the pGemT-Easy vector system (Promega). The selectable markers neomycin and a HPRT mini-gene are included along with a loxP site within the mouse sequence of the targeting vector. The flanking homology was not less than 1 kb on any one side of the construct. The second targeting construct required 6 to 10 kb of homology to the 3′ intronic sequence flanking and including the most distal constant gene sequences. A hygromycin and HPRT mini-gene were included as selectable markers along with a loxP site and were placed within the arms of homology in the order indicated, FIG. 4. The constructs were targeted to the Ig Heavy locus sequentially. Clones that have been correctly targeted at both ends of the Ig locus are transiently exposed to Cre recombinase. Deletion of the sequence intervening the loxP sites produces ES cell lines that are hemizygous for the desired deletion ie lacking one allele of the lambda locus. This cell line was used to make a homozygous KO mouse line for the Ig Lambda locus. Details of the methods for DNA ligations, vector construction ES cell transfections, ES cell selections, screening of recombinant and deleted ES clones, making KO mice and isolation of ES lines are detailed in the following sections.

ES cells harbouring the desired deletion at each of the Ig loci were used to make chimaeric mice which were subsequently bred to produce a KO mouse line containing a deletion of all 3 immunoglobulin genes.

DNA Ligation for Targeting Vector Construction

The various fragments of homolgous DNA required for the construction of the 3 targeting vectors was by long range PCR of 129 mouse genomic DNA. The ligation of DNA molecules necessary for the 3 vectors was as described in the Promega Protocols and Application Guide, (Promega). Typically vector to insert molar ratios of 1:1 and 1:3 were used for ligations. The DNA fragments were ligated into vectors which were dephosphorylated using, Shrimp Alkaline Phosphatase according to the manufactures instructions, (SAP, USB Biochemicals) Details and position of the selection cassettes relative to the immunoglobulin loci are given in FIGS. 2, 3 and 4. the ligated DNA molecules were transformed into competent DH5α E. coli cells according to the manufactures instructions (Stratagene). Bacterial colonies were recovered after overnight growth on L-agar plates at 37° C. with the appropriate antibiotic, usually ampicillin at 100 μg/ml. DNA from individual clones was isolated by the miniprep column procedure of Promega. Correctly ligated DNA molecules were identified by resolving the products of restriction enzyme analysis on 1-2% agarose gels. The desired clones were then prepped (Promega: maxi-plasmid) to produce sufficient DNA for the subsequent ligation step or ES cell transfection.

Transfecting ES Cells and Screening of ES Cells

A 100 μg of the targeting construct was linearised with the appropriate restriction enzyme according to the manufacturers instructions (New England Biolabs). The digested DNA was precipitated by the addition of 1/10 3 M sodium acetate (pH 5.2). The DNA was washed with 70% ethanol and dried at room temperature. The DNA was re-suspended with 50 μl H₂O and 50 μl 1×PBS, in a sterile environment. A large flask (T150; approximately 1×10⁸ cells) of mouse ES cells was harvested using trypsin followed by washing of the cells 3 times with 1×PBS. The construct DNA was combined with the ES cells re-suspended 1×PBS in a total volume of 800 μl. The DNA cell mix was transferred to a 0.4 cm cuvette and electroporation performed at 800 V and 3 μF using a Biorad GenePulser unit. Cuvettes stood at room temperature for 15-20 minutes post transfection and were then transferred to 5×15 cm TC plates with ES medium, 24 hours later selection was applied.

Selection and Screening of Neomycin and Hygromycin Resistant Colonies.

All cells were maintained in ES media supplemented with either G418 at 300 μg/ml or hygromycin at a concentration of 200 μg/ml. After 10-12 days of selection, colonies were picked into 24 well plates and expanded for analysis and storage. The DNA was prepared as described in Example 3. 10 μg of DNA was digested with the appropriate restriction enzyme for each sample to be screened. The DNA was run on a 0.7% agarose gel in 1×TBE and transferred by standard Southern blot techniques to a nylon membrane as described by Sambrook et al. 2001. The membrane was then probed using a P³² random prime labelled DNA probe (Stratagene). The DNA for the probe was derived from the sequence flanking the target locus but distinct from the DNA homology used in the targeting construct.

Deletion of Ig Loci by Cre Recombinase Expression and 6-TG Selection

The engineered ES cell lines with the LoxP sites flanking the Ig loci are transiently exposed to Cre recombinase and 6-TG selection to delete the floxed alleles. 1×10⁷ cells from each of the floxed ES lines were resuspended in 800 μL of 1×PBS with 25 μg of the Cre recombinase circular plasmid pOG231. The cell DNA mixture was placed in 0.4 cm cuvette and electroporated at 800 Volts 3 μF using a GenePulser (Bio-Rad). The cells were plated out onto gelatinized tissue culture plates and allowed to recover for 48 hours before 6-TG selection was applied.

The parental HM1 mouse ES cell line was derived from the 129sv/O1a that had a deletion within the Hypoxanthine PhosphoRibosylTransferase (HPRT) gene. The HM1 cell line is resistant to the purine analogue 6-thioguanine (6-TG) whereas cells expressing the HPRT gene are sensitive to this toxic analogue. The second step of the targeting strategy required the deletion of the Ig loci induced by the expression of Cre recombiase to induce the site specific recombination between the loxP sites. Removal of the Ig loci by site specific recombination also removes the 2 mini-HPRT genes. Addition of 1×6-TG kills any cells which have not removed both copies of the HPRT genes allowing recovery of those clones with a deleted allele of either the heavy, kappa or lambda Ig locus. PCR and Southern analysis of individual clones confirmed the deletion.

KO Homozygous Null Mice for the Immunoglobulin Loci

Chimaeric mice were created by blastocyst micro-injection of the 3 separate ES cell lines bearing the engineered deletion of their Ig loci either heavy or kappa or lambda. The methods used, for the blastocyst injection are described elsewhere Joyner 2000. Briefly, blastocysts were collected from 3.5 day post coitus C57BL/6J females on the morning of injection. 10-12 ES cells were microinjected into each blastocyst using a hanging drop chamber mounted on a micromanipulator. Injected blastocysts were incubated at 37° C. for a 1-2 hour recovery period. These embryos were then transferred into the lumen of the uterus of pseudopregnant foster mothers. The resulting chimaeric animals were mated with C57/bl animals and the germline offspring for the Ig deletions were identified by coat colour. The heterozygous animals (for the deletion) were bred to homozygozity by mating to their heterozygous siblings. The homozygous animals for each of the Ig deletions were maintained as inbred lines. Further inter-crossing of the single homozygous lines resulted in all 3 deletions being assimilated into a single mouse line with at least 50% 129sv genetic background. This mouse line carrying the 3 deletions provided the appropriate genetic background for the production of mouse embryos that were permissive for the isolation of ES cells using conventional technology.

Isolation of ES Cell Lines from Mouse Embryos with Inactive Ig Loci

ES cell lines were isolated from the blastocyst stage embryos. The mice used produced embryos with a genetic background that was at least 50% 129sv derived and carried homozygous null mutations for the 3 immunoglobulin loci; the Ig lambda light chain, the Ig kappa light chain and the Ig heavy chain loci. The uterine horns were removed and flushed with approximately 0.5 ml of ES isolation medium: Glasgow's Minimum Essential Medium (GMEM; Sigma) supplemented with 5% newborn calf serum and 15% fetal calf serum (Globepharm), 0.1 mM non-essential amino acids (Life technologies), 0.1 mM β-mercaptoethanol, 1 mM sodium pyruvate (Life technologies), 2 mM L-glutamine (Life technologies) and 1000 U/ml recombinant murine leukaemia inhibitory factor (ESGRO; Chemicon). Embryos were washed three times in isolation medium and explanted individually into wells of a 24-well plate, pre-seeded with mitotically inactivated STO feeder cells, Robertson E. J. 1987. The explanted blastocysts were cultured in ES isolation media for 4-6 days. The expanding inner cell mass cells of the explant were carefully removed using a finely pulled Pasteur pipette and placed into a drop of either 1×PBS or trypsin under oil. The clump of cells was gently disaggregated and seeded on to fresh inactivated STO feeder cells in the presence of ES isolation media. 5-7 days later the plates were examined for pirimary ES-like colonies. The primary ES explants were picked using pulled Pasteur pipettes and disaggregated by gentle trypsinisation into clumps and single cells, then transferred onto STO feeder layers in single wells of a 24-well plate. Approximately 1 week later each well of the 24-well plate was trypsinised and plated into a well of 6-well plate. Newly derived ES lines were removed from the isolation medium to ES maintenance medium after passage 4. The candidate ES cell lines were verified for normal karyotype and for their ability to differentiate into other cell types. The primary ES cell lines were further expanded until about passage 5 or 6 at which time cells were frozen and stored in liquid nitrogen.

### EXAMPLE 4

ES Cell Differentiation and Enrichment for HSC/Pro-B Cells.

Mouse ES cells containing Ig-YAC or human chromosome fragments from chromosomes 22, 14 and 2 were cultured and differentiated into HSC/pro-B cells or into cell fates that were committed to a B cell lineage. ES cells were grown in GMEM containing 10% FCS, 15% sodium bicarbonate, 0.1% MEM non-essential amino acids, 4 mM glutamine, 2 mM sodium pyruvate and 0.1 mM 2-mercaptoethanol in flasks coated with 0.1% gelatin. ES cells were maintained in an undifferentiated state by addition of ESGRO 1000 U/ml (Chemicon). Cultures were monitored every day to ensure that ES cells did not grow past confluence and cells were normally passaged every two days. To passage cells, culture medium was aspirated and rinsed with PBS. 1 ml Trypsin solution (0.025%, Gibco; 0.1% chicken serum Flow Labs; 1.3 mM EDTA, Sigma in PBS) for a 25 cm² flask (0.1 mls for 24 well plate, 1 mls for 75 cm² flask, 3 mls for 175 cm² flask) was added and the cells were incubated at 37° C. for 2-5 minutes until a single cell suspension was obtained. 9 mls of ES cell medium was added to to neutralise 1 ml of trypsin and the cell suspension was spun for 3 minutes at 200 g. The cell pellet was re-suspended in ES cell medium and transferred to fresh gelatinised flasks or wells at a dilution of 1:10.

A number of methods or variations of can be used to differentiate the ES cells into B cell lineage. In the first method, ES cells grown as monolayer in LIF containing medium were trypsinised and re-suspended at a concentration of 300 cells/10 μl in GMEM with LIF. Drops of 10 μl of this cell suspension were placed onto the inside of the lid of a bacteriological plate. The lid was placed on to a plate containing PBS. The cells were left in incubator for 2 days at 37° C., 5% oxygen and 7.5% CO₂. After 2 days, embryoid bodies were washed into a non-gelatinised flask or dish in medium without LIF and grown at 37° C., 5% oxygen, 7.5% CO₂ for 10-15 days. The cultures were fed every 2-3 days. Embryoid bodies were dissociated by digestion with collagenase/dispase. B220⁻/AA4.1⁺ cells were identified by FACS/MACS sorting. In the second method, the embryoid bodies were washed into a non-gelatinised flask or dish in medium without LIF but supplemented with 5 ng/ml Il-7. The embryoid bodies were grown for 7 days with medium changed as appropriate and then plated onto mitomycin-C treated ST-2 cells in media supplemented with 5 ng/ml Il-7. The embryoid bodies were grown for 5 days and then the non-adherent cells were removed onto mitomycin-C treated ST-2 cells with 5 ng/ml Il-7. B220⁻/AA4.1⁺ cells were isolated from non-adherent cells by FACS/MACS sorting after a further 7 days of culture.

Mouse ES cells were also differentiated to mature B cells on OP9 cells. OP9 cells were cultured in α-MEM medium supplemented with 20% FCS and 2.2 mg/ml sodium bicarbonate. Co-cultures of OP9 and ES cells were also grown in α-MEM containing 20% FCS and 2.2 mg/ml sodium bicarbonate. ES cells were plated on OP9 cells growing in 6 well plates and fed with fresh medium on day 3. On day five, the cells were trypsinised and preplated for 30 min. Non-adherent cells were plated onto OP9 cells growing on 10 cm plates at 1-2×10⁶ per plate with 20 ng/ml Flt-3. On day 8, non-adherent cells were collected and plated onto fresh OP9 cell layers with 20 ng/ml Flt-3. Cells were cultured for a further 7 days with a medium change at day 12. The cells were harvested at day 15, plated onto fresh OP9 cells and cultured for a further 13 days. On day 19, the cells were analysed for IgM and B220 expression by FACS. On day 28, the cells were stimulated with 100 μg/ml LPS for 48 hours and the cells were analysed for up regulation of CD80 by FACS.

Another approach used was based on the procedures of Miyagi et al., (2002), Experimental Hematology, 30, 1444-1453. “Flk1⁺ cells derived from mouse embryonic stem cells reconstitute haematopoiesis in vivo in SCID mice”. Briefly, undifferentiated ES cells were cultured on non-coated tissues in Iscove's modified Dulbecco's medium (Invitogen) supplemented with 0.8% methylcellulose, 15% fetal calf serum and 450 μM thioglycerol for 4 days. The large cell aggregates were collected and dissociated in to single cell suspension using phosphate-buffered saline and 1 mM EDTA. The Flk1⁺ cells were collected by autoMACS sorting (Mitlenyi Biotec), for this the phycoerythrin (PE)-conjugated anti-mouse monoclonal antibody and anti-PE microbeads (Miltenyi Biotech) were used.

The preferred method of deriving HSC cells from the humanised ES cells was based on the methods described by Burt et al., 2004. Briefly, To induce differentiation toward HSCs in vitro, the ES cells were cultured on low adherent Petri dishes in Iscove's modified Dulbecco's medium containing 1% methylcellulose, 15% FBS, 150 M monothioglycerol, 2 mM glutamine, 500 ng/ml murine SCF, 46 ng/ml human IL-3, and 500 ng/ml human IL-6 (StemCell Technologies Inc. and Sigma-Aldrich) Cells were cultured at 37° C. in 5% CO₂ atmosphere incubator for 7-10 d. The single cell suspension was collected, washed, and suspended in 1×PBS at a concentration of 10⁷ cells/0.2 ml for i.v. injection or 0.5×10⁷ cells/30 μl in the case of intra bone marrow (IBM) injection.

HSC/pro-B cells were isolated from mouse bone marrow (BM) using Magnetic antibody cell sorting (MACS) and fluorescence activated cell sorting (FACS). Mouse BM was aspirated from femurs with 26 gauge needle and place directly on ice. The cell suspension was treated with 6 ml red cell lysis buffer (155 mM NH₄Cl; 10 mM KHCO₃; 0.1 mM EDTA) for 5 minutes at room temperature and washed twice with 1% BSA in PBS. Cells were sorted into AA4.1⁺/B220⁺ double positive cells using anti-FITC multisort kit and anti-B220 microbeads according to manufacturers instructions (Miltenyi Biotec Ltd) or into mAA4.1+ cells using anti-FITC microbeads according to manufacturers instructions (Miltenyi Biotec Ltd.) AA4.1⁺/B220⁺ population represents approximately 10% of total red cell lysed BM cells. For FACS analysis, mouse BM cells were prepared or cells were initially sorted by MACS. Cells in a single cell suspension were counted and 2×10⁵ cells were placed in a well of a 96 well plate. The plates were spun at 900 rpm for 1 min and the supernatant was removed. The cells were resuspend in 100 μl of 1% BSA in PBS containing 0.2 μg of the appropriate antibody or antibodies and incubated at 4° C. for 30 mins, centrifuged at 900 rpm for 1 min and washed 3 times with 1% BSA in PBS. For secondary staining with strepdavidin-conjugated flourochromes, the cells were resuspended in 100 μl of 1% BSA in PBS containing Strepdavidin-PE at a dilution of 1:50 or Extravidin-FITC at a dilution of 1:200. The samples were incubated at 4° C. for 30 minutes and washed as above. After the final staining, the cells were re-suspended in 200 μl 1% BSA in PBS for direct analysis or 200 μl 1% BSA in PBS containing 0.025% paraformaldehyde and 0.01% sodium azide if cells were to be analysed more than 12 hours later. The antibody-stained cells were stored at 4° C. in the dark until analysis within 5 days. FACS analysis was performed on Becton Dickinson FACScan Machine. FITC and PE labeled cells were identified in the FL-1 and FL-2 channels respectively. Analysis by FACS showed up to 90% pure AA4.1⁺ cells after single-sorting and up to 97% pure AA4.1⁺/B220+ cells after multi-sorting with anti-FITC multisort kit with MACS.

AA4.1⁺/B220⁺ cells isolated as above were also grown on subconfluent mitomycin-C treated ST-2 BM stromal cell line with addition of rIl-7 (5 ng/ml) (Sigma). ST-2 cells were maintained in DMEM supplemented with 10% FCS. ST-2 cells were treated with mitomycin-C (10 μg/ml) for 3 hours. The mitomycin-C treated cells were washed 3 times in PBS and plated at 6×10³-1.2×10⁴/well of 96 well plate.

Another method for the commitment of ES cells to the hematopoetic lineage was provided by Kennedy and Keller (2003). This method required the ES cells be grown in specialised methyl cellulose containing media conditions in the absence of LIF for the formation of embroid bodies (EBs). EBs can be harvested at different stages of development and examined for their hematopoeitic potential by growing in methylcellulose cultures with selected cytokines. Details of such methods are included within.

Generation of Embryoid Bodies

Prior to initiating EB development, it was necessary to deplete the ES culture of feeder cells, as they will alter the kinetics of differentiation if present in the EB cultures. Feeder depletion was accomplished by passaging the cells two times on gelatin-coated wells in the presence of LIF. This was a critical step as most feeder-dependent ES cells differentiate when moved to a gelatin-coated surface. To minimize the extent of differentiation, the ES cell density was kept high when passaging without feeders. To ensure appropriate cell density, we used 2-3 different dilutions (ranging from two- to six-fold) and selected the well with the appropriate concentration of cells for the next passage. The appropriate concentration contained a high density of undifferentiated ES cell colonies. The ES cell colonies retained their typical three-dimensional structure with well-defined smooth perimeters. Cells were passaged at 24-hr intervals and maintained at high density. For hematopoietic differentiation of the ES cells, the first feeder depletion passage was done in DMEM-ES medium, whereas the second was done in IMDM-ES medium. Cells were passaged twice in the absence of feeders and used for the generation of EBs. Depleted ES cells were trypsinised and wash two times with IMDM-FCS (no LIF) to remove residual LIF from the medium. EBs were generated in non-adherent 60 mm Petri grade dishes. The number of ES cells plated in the differentiation cultures depended on the cell line used and the progenitor population assayed. It was important to test different cell numbers for the particular ES cell line being used as too few or too many cells resulted in suboptimal differentiation. Optimal conditions produced 1.5×10⁶ to 3.0×10⁶ cells per 60 mm dish. In addition to cell yield, differentiation was monitored by levels of expression of the receptor Flk-1 and the number of blast colonies at the hemangioblast stage of development (days 2.5-4.0). Hematopoietic stage EBs (day 6-7 of differentiation) were assayed for hematopoietic progenitor potential. It is important to note that the cell numbers and the components of the medium used for the generation of the two stages of EB development were different. Media used for hemangioblast expansion: 10% FCS, 10% Horse serum, 5 ng/ml VEGF, 10 ng/mlIGF-1, 10/ng/ml bFGF, 2 units/ml Epo, 100 ng/ml KL, 1 ng/mL-3, 1.5×10⁴ M MTG, IMDM supplemeted to 100%. Media used for the in vitro differentiation of numerous cell types from EBs: 55% methylcellulose, 10% PDS or 5% FCS, 3.0×10⁻⁴ M MTG, 25 ng/ml ascorbic acid, 2 mM glutamine, 300 ug/ml transferring, 100 ng/ml KL, 2 Units/ml Epo, 5 ng/ml Tpo, 1 ng/ml IL-3, 10/ng/ml 11-6, 5 ng/ml IL-11, 3 ng/ml GM-CSF, 30 ng/ml G-CSF, 5 ng/ml M-CSF, IMDM up to 100% and various combinations of the above cytokines.

Analysis of EBs and Harvesting of EBs

Harvested EBs from the differentiation cultures were placed in a 50 ml tube and allowed to sit for for approximately 5-10 min so as the EBS would settle to form a loose pellet. A maximum of eight dishes were pooled into a single 50 ml tube. The supernatant was removed and 3 ml of trypsin was added to the EBs and placed in a 37° C. water bath for 3 min. 1 ml of FCS was added to the trypsin and vortexed, to dissociate the EBs. After the dissociation process, the cells were passaged through a 20-gauge needle 1-2 times, using a 5 ml syringe and transfered to a 14 ml tube containing 6 ml of IMDM-FCS. Centrifuged and resuspend in 1-2 ml IMDM-FCS, using a P1000 pipette. The EB cells were counted and analyzed for hemangioblast and hematopoietic potential.

Analysis of Hemangioblast and Hematopoietic Potential of EBs

The hemangioblast stage of development precedes the onset of hematopoiesis within the EBs and is found between days 2.5 and 4.0 of differentiation for most ES cell lines. The BL-CFC represents a transient population that is found in the EBs for only 18-36 hr. Consequently, any changes in culture conditions that alter the kinetics of development by as little as 6 hr, can lead to inconsistencies between experiments. Between days 2.5 and 4.0 of differentiation during which time the number of secondary EBs is declining. Secondary EBs develop from residual ES cells that have not yet differentiated in the primary EBs. Beyond day 4.0 of differentiation, the number of BL-CFC declines with the commitment to the hematopoietic program was indicated by the appearance of significant numbers of primitive erythroid progenitors. Given that the BL-CFC expresses the receptor tyrosine kinase Flk-I, its development is associated with the up regulation of receptor expression within the EBs. These dynamic changes in Flk-1 expression are the expected pattern and indicative of efficient and appropriately timed differentiation. To minimize any changes in the kinetics of differentiation, it is important to adhere strictly to the protocol, using the same reagents for each experiment. While the presence of Flk-1 within an EB population does not guarantee the presence of large numbers of BL-CFCs, the lack of significant Flk-1 expression does indicate that the population has not yet progressed to the hemangioblast stage of development. Expression of Flk-1 can be used as an initial screen to define the appropriate day of EB development for BL-CFC analysis in subsequent experiments.

For FACS analysis, antibody staining was carried out as follows: 2×10⁵ cells were resuspended in 100 μl of PHS containing 10% FCS and 0.02% sodium azide. An appropriate amount of antibody was added and the cells were incubated on ice for 20 min. Following the staining step, the cells were washed twice with the same media. To assay for the BL-CFC, an aliquot of the cell suspension with the appropriate number of cells for 3.5 ml media was place in a 14 ml snap cap tube. The volume of the cell suspension did not exceed 10% of the total mixture. Larger volumes reduced the viscosity of the methylcellulose mixture. 3.5 ml of the hemangioblast colony methylcellulose mixture (55% methylcellulose 10% FCS, 3×10⁴ M MTG, 25 ng/ml Ascorbic acid 2 mM glutamine 300 ug/ml transferring 25% D4T CM, 5 ng/ml VEGF, 10 ng/ml IL-6, up to 100% IMDM) was added to the cells using a syringe with a 16-gauge needle. It was important to have more methylcellulose (approximately 0.5 ml) than is actually needed as it was impossible to recover all of the mixture from the tube. The mixture was vortexed with the cells and then allow to settle for 5 min to permit the air bubbles to rise. 1 ml of the mixture was aliquoted into each of 3×35 mm petri grade dishes. The dishes were gently shaken to disperse the methylcellulose mix evenly over the bottom surface. The dishes were placed in a larger dish, together with a 35 mm open dish of water for humidity and incubated at 37° C. in an environment of 5% CO₂ in air. For most ES cell lines, we used between 3×10⁴ and 1×10⁵ cells per ml for BL-CFC analysis. The frequency of the BL-CFC ranged from 0.2 to 2.0% of the total EB population. Blast colonies developed within 3-4 days of culture and can be recognized as clusters of cells that are easily distinguished from secondary EBs that develop from residual undifferentiated ES cells. Blast colonies and secondary EBs were the predominant type of cells and were harvested by gentle pipetting and assayed for hematopoietic progenitor potential in methylcellulose cultures using the multi-lineage (mix 5: 55% methylcellulose, 10% FCS, 5% PFHM-II, 3×10⁻⁴ M MTG, 25 ng/ml Ascorbic acid 2 mM glutamine 300 ug/ml transferring, 100 ng/mlKL, 2 Units/ml EPO, 5 ng/ml TPO, 1 ng/ml IL-3, 10 ng/ml IL-6, 5 ng/ml IL-11, 3 ng/ml GM-CSF, 30 ng/ml G-CSF, 5 ng/ml M-CSF and up to 100% IMDM) that supported the growth of multiple progenitors. The remaining adherent population was cultured for an additional 4 days in endothelial expansion medium. At this point, the adherent population was lysed directly in the well and subjected to RT-PCR for the analysis of expression of genes associated with endothelial development.

Hematopoietic Stage

Shortly following the peak of the hemangioblast stage of development, committed hematopoietic progenitors were detected within the EBs. Both primitive erythroid and definitive hematopoietic progenitors appeared as early as day 4 of differentiation. Following the initiation of primitive erythropoiesis between days 3.5 and 4.0 of differentiation, this progenitor population increased in size dramatically, reaching a peak by day 6: Primitive erythroid progenitor numbers droped sharply to undetectable levels over the next 48 to 72 hr. The number of definitive progenitors increased at this stage and reached a plateau between days 6 and 7 of differentiation. While defnitive progenitor numbers declined beyond this point, the decrease is not as dramatic as observed with the primitive erythroid lineage. It is worth stressing that these patterns are based on EBs differentiated in medium containing PFHM-II and serum pre-selected for optimal differentiation to the hematopoietic lineages. PFHM-II was included in these cultures as we have found that it increased the efficiency of EB differentiation to hematopoiesis. It was not included in the EB hemangioblast medium, however, as it reduced the number of detectable BL-CFC, possibly due to acceleration of the kinetics of differentiation at this stage. All wild type ES cell lines that we have tested follow this pattern of development. To evaluate the primitive and definitive hematopoietic potential of a given cell line, we routinely analyzed EBs differentiated for 6 days. For this analysis, the EBs are harvested and processed as described above. The EB-derived cells were plated in methylcellulose media containing different hematopoietic cytokines. The selection of the media will depend on the type of progenitor to be analyzed. While the large mix of cytokines present in mix 5 stimulated progenitors of all the lineages present within the day 6 EBs, at times it was desirable and advantageous to evaluate the growth of a selected subset of hematopoietic lineages, (mix 5: 55% methylcellulose, 10% FCS, 5% PFHM-II, 3×10⁻⁴ M MTG, 25 ng/ml Ascorbic acid 2 mM glutamine 300 ug/ml transferring, 100 ng/ml KL, 2 Units/ml EPO, 5 ng/ml TPO, 1 ng/ml IL-3, 10 ng/ml IL-6, 5 ng/ml IL-11, 3 ng/ml GM-CSF, 30 ng/ml G-CSF, 5 ng/ml M-CSF and up to 100% IMDM)

A further method of lymphoid cell isolation from mouse ES cells was provided by Fraser et al. 2004. The details of which are summarised as follows.

Induction and Purification of Lateral Plate Mesoderm from ES Cells

Undifferentiated ES cells were split by brief trypsinization. 1×10⁴ undifferentiated ES cells was added to each well of a 6-well collagen-coated dish. 3 ml of differentiation medium, (Alpha modified Eagle's medium containing 2-ME, Pen/Strep and 10% FCS from Gibco BRL), was added to each well. The culture was undisturbed for 4 days in 5% CO₂ 37° C. environment. Cells were trypsinised for 10-20 min until the differentiated cells began to dislodge from the bottom of the well. Harvested cells were incubated with NMS for 10-20 min on ice to block non-F(Ab) specific interactions. Fluorescently conjugated rat anti-mouse Flk1 and rat anti-mouse E-cadherin MAbs were incubated with the cells on ice for 20 min. Cells were washed twice with PBS. Cells were resuspended in HBSS/BSA/PI. Flk1⁺ E-cadherin⁻ population of cells was isolated by flow cytometric isolation.

Differentiation and Isolation of ES-Derived Endothelial Cells

Flk1+ cells were sorted as decribed above. 1×10⁴ cells/well was added to a 6-well culture dish containing confluent OP9 cells with 3 ml differentiation medium (Alpha modified Eagle's medium containing 2-ME, Pen/Strep and 10% FCS from Gibco BRL). Culture was left undisturbed for 3 days. Harvest cells and prepare for antibody staining as described in section “Induction and Purification of LPM from ES Cells.” Cells were incubated with fluorescently labelled anti-mouse VE-cadherin mAb for 20 min on ice. Cells prepared for sorting as described in section “Induction and Purification of LPM from ES Cells.” Sort VE-cadherin+ cells using flow cytometer. The endothelial cells were added to confluent cultures of OP9 stromal cells. Differentiation medium containing hematopoietic growth factors awasd added. To encourage erythroid-myeloid lineages SCF, Epo and IL-3 were added. B lymphoid cells were generated by culture with differentiation medium containing SCF, Flt3 ligand and IL-7, that was changed every 3-4 days. The hematopoietic cells were harvested and analysed by flow cytometry using fluorescently conjugated MAbs recognizing lineage-specific markers. Erythropoiesis occured within 3-4 days. Myeloid cells were detected from 4 to 7 days while B lymphoid cells required approximately 2 weeks of culture.

Another strategy required the viral transduction of hematopoetic cultures with the Bcr/Abl oncogene to drive proliferation of the HSC cells, Kyba et al. 2003, the method is summarised as follows. EBs were generated by plating 10⁴ ES cells per ml in differentiation medium {IMDM (Sigma) with 15% fetal calf serum (FCS for differentiation; StemCell), 50 ug/ml ascorbic acid (Sigma), 200 ug/ml iron-saturated transferrin (Sigma), 4.5×10⁻⁴ M monothioglycerol (MTG; Sigma)} supplemented with 0.9% methylcellulose (M3120, StemCell Technologies) in 35 mm petri dishes (StemCell). Harvest on day 5 (120 hr) by diluting the methylcellulose with PBS and centrifuging the EBs. Washed once with PBS, and dissociated with ˜0.25% collagenase for 60 min at 37° C. followed by repeated passage through a 23 G needle. Cells were infected by spinning (1300 g for 90 min at 30° C. in a Beckman GH-3.8 rotor) 3×10⁵ cells with 10 ml of Bcr/Abl viral supernatant containing 4 μg/ml polybrene (Sigma) in three wells of a 6-well dish precoated with stromal cells. Transduced cells were cultured on OP9 stroma at 37° C. with 5% CO₂. The growth conditions under which transduced populations are initiated are critical. We used a cytokine cocktail consisting of 0.5 ng/ml murine IL3 (interleukin 3; Peprotech), and 50 ng/ml each of human IL6 (interleukin 6; Peprotech), human SCF (stem cell factor; Peprotech), and human FL (FLT3 ligand; Peprotech), 50 μM {3-mercaptoethanol, in IMDM/15% FCS, over OP9 stromal cells. Once the colonies had become dense, they were passaged in the absence of stroma in the same growth medium. Under these conditions, cultures became dominated by immature hematopoietic blast cells.

### EXAMPLE 5

Transplantation of HSCs Cells into Immuno-Compromised Mice.

HSC/Pro-B Cell Containing Human Ig Loci were Either Intravenously Transplanted or Introduced by Intra Bone Marrow Injection into Irradiated Mouse.

HSC/pro-B cells were isolated from ES cells by culture regimes described in Example Cells were resuspended in ice-cold PBS at appropriate concentrations with 2×10⁴ adult bone marrow cells for each transplant as short-term radio-protection. Recipient mice were irradiated at 9.5 Gr (split into two doses separated by a 3 hour interval in the Cs source at a rate of 21.6 rad/min). Cells were transplanted via tail vein injection into the lethally irradiated recipient mice. The volume of cell suspension transplanted should not exceed 400 μl per recipient. Recipient mice received neomycin (0.16 g/100 ml) in acid drinking water for the first four weeks after irradiation and were housed in microisolator cages under specific pathogen-free conditions and provided with irradiated food. The recipient mice were 6-7-wk-old female BALB/cJ mice (MHC H2 d; Jackson Laboratories).

The preferred method of transplantation required an enriched population of HSCs collected by FACS purification and then transplanted to recipient animals by intra bone marrow (IBM) injection. The injection was performed as described by Kushida et al., 2001. The region from the inguen to the knee joint was shaved of hair with a razor and a 5-mm incision was made on the thigh. The knee was flexed to 90 and the proximinal side of the tibia was drawn to the anterior. A 26-guage needle was inserted into the joint surface of the tibia through the patellar tendon and then inserted into the bone marrow cavity. Using a microsyringe (50 μl; Hamilton) containing the donor HSC (3×10⁷/30 μl), the donor cells were injected from the bone hole into the bone marrow cavity. The skin was then closed using 6-0 vicryl sutura. Alternatively the cells were transplantation by intra-venous injection into one of the lateral tail veins.

### EXAMPLE 6

Expression Analysis of Human Ig Genes.

The expression of human antibodies in the sera of the chimaeric mice transplanted with the the humanised ES cells for the heavy and light immunoglobulin loci can be detected by enzyme-linked immunosorbent assay (ELISA). The standard methods used were essentially those outlined in “Antibodies a Laboratory Manual” edited by Ed Harlow David Lane, published by Cold Spring Harbour 1988. Chimaeric mice from Example 4 were immunised with human serum albumin (HSA, Sigma). To determine antigen-specific human antibodies, ELISA was performed using recombinant human TNFα and MAGE1, MAGE2 and MAGE3. The concentrations of human and mouse immunoglobulins were analysed using antibodies against human IgG. Igλ and IgM, and mouse IgG, Igλ, Igκ and IgM respectively (Sigma, Southern Biotechnology and Vector). Detection antibodies were conjugated with HRP or biotin.

Ig Gene Rearrangement

The expression of human Ig genes was also analysed at the RNA level by RT-PCR. Poly A⁺ RNA was prepared from peripheral blood lymphocytes using the RNeasy Isolation kit from Quiagen Inc. The cDNA synthesis was performed using the first strand synthesis kit of Pharmacia. Primers used in RT-PCR are listed below.

RNA Isolation and cDNA Synthesis

Approx 1×10⁶ cells were harvested for RNA extraction, to this was added 350 μl of RLT solution and the sample vortexed for 30 s. Lysate was centrifuged for 3 min at maximum speed. To the isolated supernatant was added 1 vol (350 μl) of 70% ethanol. The solution was then applied to an Rneasy column sitting in a 2 ml collection tube and centrifuge for 15 sec at 8000 g. The column was washed twice with 500 μl of buffer RPE. The RNA was eluted from the column with 50 μl of Rnase-free H2O. percipitate DNA by addition of 0.1 vol (5 μl) 3M sodium acetate ph 5.2 add 2 vol ice cold ethanol (110 μl) and 2 μl of seeDNA (Amershm) to trace the RNA. The sample was precipitated by the addition of ethanol and spun to pellet the RNA. The pellet was then resuspended in 8 μl water. The 8 μl sample of RNA was heated to 65° C. for 10 min. The following was added to the sample: 5 μl of Bulk 1st Strand Mix, 1 μl DTT, 1 μl d(N₆) primer and incubated at 37° C. for 1 hour. The newly synthesised cDNA was aliquoted and stored at −70° C. until required.

Identification of Ig Gene Rearrangements by RT-PCR

The methods used were similar to those described in Nicholson et al. 1999.

Briefly the primer sets used were as follows:

Heavy chain family leaders (sense, codons −20 to −13) V_{H}1 leader; 5′-atg gac tgg acc tgg ag-3′, V_{H}2 leader. 1; 5′-atg gac ata ctt tgt tcc acg c-3′, V_{H}2 leader. 2; 5′-atg gac aca ctt tgc tcc acg c-3′, V_{H}4 leader; 5′-atg aaa cac ctg tgg ttc ttc-3′, V_{H}6 leader; 5′-atg tct gtc tcc ttc ttc-3′, Heavy chain constant (antisense, codons 127-134) IgM constant; 5′-cgt atc cga cgg gga att ctc aca-3′, Heavy chain consensus framework 1 (sense, codons 1-8) Universal VH; 5′-gag gtg (ac)a(ag) ctg cag (cg)ag tc(at) gg-3′, Heavy chain consensus framework 4 (antisense, codons 103-111) Heavy joining; 5′-cag ggt gac cag ggt acc ttg gcc cca g-3′, κ light chain consensus framework 1 (sense, codons 1-8) Vκ all 5′-ga(ac) a(ct)(ct) gag ctc acc cag tct cca-3′ κ light chain constant (antisense, codons 112-119) κ constant; 5′-cgg gaa gat gaa gac aga tgg tgc-3′, κ light chain consensus framework 4 (antisense, codons 101-108) κ join; 5′-g ttt gat ctc cag ctt ggt ccc-3′, λ light chain family framework 1 (sense, codons 1-7) Vλ2; 5′-cag tct gcc ctg act cag cct-3′, λ light chain family framework 1 (sense, codons 1-7) Vλ3; 5′-tcc tat gag ctg ac(at) cag-3′ λ light chain constant (antisense, codons 126-132) λ constant; 5′-cg tgt ggc ctt gtt ggc t-3′, λ light chain consensus framework 4 (antisense, codons 101-106a) λ join; 5′-tag gac ggt (cg)a(cg) ctt ggt ccc-3′.

The amplified rearrangements were purified using the QIAquick Prep System and cloned into the pGem-T vector (Promega UK) Recombinant clones were screened for appropriate rearrangements by PCR using the primers to the respective framework 1 and framework 4 detailed above. Plasmid was isolated and sequenced using standard M13 forward or reverse sequencing primers

### EXAMPLE 7

Generation of Hybridoma.

Hybridoma cell lines were prepared and assessed for human antibody production by methods essentially outlined in “A practical guide to monoclonal antibodies” edited by Liddel and Cryer, published by John Wiley and Sons 1991 or “Antibodies a Laboratory Manual” edited by Ed Harlow David Lane, published by Cold Spring Harbour 1988. Myeloma cells such as MOPC-21 derived and NSO-bc12 cells were fused with splenocytes isolated from chimaeric mice, transplanted with the engineered ES-hIg cells, expressing human immunoglobulins to make hyridoma cells. Myeloma cells were cultured in RPMI 1640 medium with 10% FCS in a 5% CO₂ and 37° C. incubator. A splenocyte suspension was prepared from the chimaeric mice expressing human immunoglobulins. The mouse was sacrificed and swabbed with 70% ethanol. The spleen was removed and placed in sterile saline. The tissue was transferred into a petri dish. 10 ml culture medium was injected into the spleen at multiple sites with a 25-g needle. The cells dispersed into the medium were transferred into a sterile tube and spun at 1500 rpm on a bench centrifuge for 10 min. The cell pellet was washed with 10 ml serum free medium and resuspended in 5 ml serum free medium. Polyethylene glycol (PEG) was used to fuse the myeloma cells with the splenocytes. Myeloma cells were collected and washed with serum free medium. 10⁸ myeloma cells and 10⁸ splenocytes were mixed and pelleted at 1500 rpm for 5 min. 1 ml PEG 1500 was added to the pellet slowly and left for 1 min. 20 ml serum free medium was added slowly and left at room temperature for 10 min. After two washes in serum free medium, cells were distributed in 5 96-well plates and incubated at 37° C. overnight. HAT was applied. Screen for specific antibody production from hybrids was performed according to standard procedures for the production of monoclonal antibodies.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry, molecular biology and biotechnology or related fields are intended to be within the scope of the following claims.

### REFERENCES

Battista S, Pentimalli F, Baldassarre G, Fedele M, Fidanza V, Croce C M, Fusco A. Loss of Hmga1 gene function affects embryonic stem cell lympho-hematopoietic differentiation. FASEB J. 2003 17(11): 1496-8. Epub 2003 Jun. 17.
Bruggemann M. Human antibody expression in transgenic mice. 2001 Arch Immunol Ther Exp (Warsz). 2001;49(3):203-8. Review.
Dorshkind K, Keller G M, Phillips R A, Miller R G, Bosma G C, O'Toole M, Bosma M J. Functional status of cells from lymphoid and myeloid tissues in mice with severe combined immunodeficiency disease. J. Immunol. 1984; 132(4):1804-8.
Frazer S., Yanishata J., Jakt L., Okada M., Ogawa M., Nishikawa S., Nishikawa S. In Vitro Differentiation of Mouse Embryonic Stem Cells: Hematopoietic and Vascular cell types. Methods Enzymol. 2003; 365: 59-72
Green L, L. Antibody engineering via genetic engineering of the mouse: XenoMouse strains are a vehicle for the facile generation of therapeutic human monoclonal antibodies. J Immunol Methods. 1999 10;231(1-2):11-23.
Harlow, E. and Lane D. Antibodies a Laboratory Manual, published by Cold Spring Harbour 1988.
Ishida I, Tomizuka K, Yoshida H, Tahara T, Takahashi N, Ohguma A, Tanaka S, Umehashi M, Maeda H, Nozaki C, Halk E, Lonberg N. Production of human monoclonal and polyclonal antibodies in TransChromo animals. Cloning Stem Cells. 2002;4(1):91-102. Review.
Joyner A. J. Gene Targeting: A Practical Approach. IRL Press, Oxford, 2000
Kennedy M. and Keller G. M. Hematopoetic Commitment of ES cells in Culture. Methods Enzymol. 2003; 365:39-59.
Kushida T., Inaba M., Hisha H., Ichioka N., Esumi T., Ogawa R., Iida H., and Ikehara S. Intra-bone marrow injection of allogeneic bone marrow cells: a powerful new strategy for treatment of intractable autoimmune diseases in MRL/lpr mice. Blood 2001, Vol 97: 3297-3299.
Kyba M., Perlingerio R. C. R. and Daley G. D., development of Hematopoietic repopulating cells from embryonic stem cells. Methods in Enzymology. 2003, 365: 114-119.
Lee J. T, Jaenisch R. A method for high efficiency YAC lipofection into murine embryonic stem cells. Nucleic Acids Res. 1996;24(24):5054-5.
Liddel E. J., and Cryer A., A Practical Guide to Monoclonal Antibodies, published by John Wiley and Sons, 1991.
Miyagi T, Takeno M, Nagafuchi H, Takahashi M, Suzuki N. Flk1+ cells derived from mouse embryonic stem cells reconstitute hematopoiesis in vivo in SCID mice. Exp Hematol. 2002;30(12):1444-53.
Mombaerts et al. RAG-1-deficient mice have no mature B and T lymphocytes. Cell. 1992. 68(5):869-77.
Robertson E. J. Embryo derived ES cells. In Tetracarcinomas and embryonic stem cells a practical approach. (ed. Robertson, E. J.) 71-112 (IRL Press, Oxford, 1987).
Sambrook J. and Russel W. D. Molecular Cloning: A Laboratory Manual. 2000
Shinkai et al. RAG-2-deficient mice lack mature lymphocytes owing to inability to initiate V(D)J rearrangement. Cell. 1992. 68(5):855-67.
Tanabe, H., Nakagawa, Y., Minegishi, D., Hshimoto, K., Tanaka, N., Oshimura, M., Sofuni, T., and Mizusawa, H. Human monochromosome hybrid cell panel characterized by FISH in the JCRB/HSRRB, Chromosome Res., 2000, 8: 319-334.
Tomizuka K, Yoshida H, Uejima H, Kugoh H, Sato K, Ohguma A, Hayasaka M, Hanaoka K, Oshimura M, Ishida I. Functional expression and germline transmission of a human chromosome fragment in chimaeric mice. Nat Genet. 1997 June;16(2):133-43.
Tomizuka K, Shinohara T, Yoshida H, Uejima H, Ohguma A, Tanaka S, Sato K, Oshimura M, Ishida I. Double trans-chromosomic mice: maintenance of two individual human chromosome fragments containing Ig heavy and kappa loci and expression of fully human antibodies. Proc Natl Acad Sci USA. 2000 18; 97(2): 722-7.
Thompson S, Clarke A R, Pow A M, Hooper M L, Melton D W. Germ line transmission and expression of a corrected HPRT gene produced by gene targeting in embryonic stem cells. Cell. 1989 27;56(2):313-21.
Williams S C, Winter G. Cloning and sequencing of human immunoglobulin V lambda gene segments. Eur J Immunol. 1993;23(7):1456-61.
Zhang Z, Cotta C V, Stephan R P, de Guzman C G, Klug C A.
   Enforced expression of EBF in hematopoietic stem cells restricts lymphopoiesis to the B cell lineage. EMBO J. 2003 Sep. 15; 22(18): 4759-69.
Huxley, C. and Gringe, A. Transfer of Yeast artificial chromosomes from yeast to mammalian cells. Bioessays. 1991. 13: 545-550.
Lee T. and Jaenisch R. A method for high efficiency YAC lipofection into murine embryonic stem cells. NAR, 1996, Vol 24, 24
Marschall P., Malik N. and Larin Z. Transfer of YACs up to 2.3 Mb intact into human cells with polyethlenimine. Gene Therapy. 1999, 6(9):1634-7.
Doherty A M, Fisher E M. Microcell-mediated chromosome transfer (MMCT): small cells with huge potential. Mamm Genome. 2003. 14(9):583-92.
Monard S P. Chromosome sorting and analysis by FACS. Methods Mol Biol. 1998;91:239-54

1. A method for the generation of a heterologous immunoglobulin molecule from a non-human somatic transgenic animal which method comprises the steps of:
(a) Creating one or more non-human somatic transgenic animal/s (i) by transfecting suitable donor cells with nucleic acid encoding a heterologous immunoglobulin molecule, (ii) transplanting said cells into a host animal; wherein the native immune system of those recipient host animal/s is suppressed and/or functionally inactivated (thus generating a non-human somatic transgenic animal); and
(b) Expressing one or more heterologous immunoglobulin molecules from one or more cells of those host animals treated according to step (a).

2. The method for the generation of a heterologous immunoglobulin molecule from a non-human somatic transgenic animal according to claim 1 which comprises the additional steps of: commitment of donor cells to a B cell developmental fate prior to transplantation into the host animal.

3. The method according to claim 2 which comprises the additional step of the enrichment of committed donor cells prior to their transplantation into host animals.

4. The method according to claim 2 wherein the donor cells are any one or more of those cells selected from the group consisting of the following: ES cells including ES-like cells, embryonic germ cells (EG cells), bone marrow cells, B-cells, multipotent progenitor cells, and MAPCs derived from adult bone marrow, and pluripotent cells derived from somatic tissue.

5. The method according to claim 2 wherein the donor cells are ES cells including ES-like cells and they are differentiated into one or more cell types prior to transplantation into host/recipient animals.

6. The method according to claim 3 wherein committed donor cells are enriched using the sorting techniques of magnetic activated cell sorting (MACs) and/or FACs sorting.

7. The method of claim 1 wherein the endogenous Ig locus of those host cells has been functionally inactivated by gene targeting.

8. The method of claim 1 wherein nucleic acid encoding one or more immunoglobulin molecules is transfected into said donor cell by the use of one of the following vectors:
(a) a fragment of a chromosome containing a complete Ig locus
(b) a human artificial chromosome vector containing a complete Ig locus
(c) a YAC vector containing a complete Ig locus
(d) a fragment of a chromosome, human artificial chromosome or YAC containing at least a portion of an Ig locus.

9. The method according to claim 8 wherein the Ig locus is a human Ig locus.

10. The method according to claim 9 wherein donor cells are transfected with nucleic acid encoding a heterologous immunoglobulin molecule using a method from the list consisting of the following: electrophoration, micro-injection, liposomal transfer and viral transduction.

11. The method according to claim 9 wherein step (b) of claim 1, that is the step of expressing one or more heterologous immunoglobulin molecules from one or more cells of the host animal comprises the establishment of one or more cell lines from one or more of those host animals and the expression of heterologous immunoglobulin molecule therefrom.

12. The method of claim 1, wherein the host animal animal is any one of those animals in the group consisting of: rodent, guinea-pig, rabbit, camelid, pig and shark.

13. The method according to claim 12 wherein the rodent is a mouse.

14. The method of claim 1 wherein the endogenous/native immune system of the host animal is suppressed and/or functionally inactivated by the use of either:
a) radiation
b) antisense RNA
c) siRNA
d) anti-endogenous Ig chain antibodies
e) chemical methods.

15. The method according to claim 14 wherein the host animal is a RAG mouse or a SCID mouse.

16. The method of claim 1 wherein the heterologous immunoglobulin molecule is an antibody molecule.

17. The method according to claim 16 wherein the antibody molecule is any of those in the group consisting of the following: monoclonal antibody; polyclonal antibody; dAb, scFv and Fab.

18. The method according to claim 17 wherein the antibody is monoclonal.

19. The method according to claim 17 wherein the antibody molecule is fully/entirely human.

20. The method according to claim 19 wherein the antibody molecule is partially human.

21. A method for generating a plurality of B cells expressing antibody molecules, the method comprising the step of providing a non-human transgenic host animal generated according to claim 1 and immunising said host animal.

22. The method of claim 21, wherein the plurality of B cells expresses one or more antibodies selected. from the group consisting of: IgA, IgD, IgE, IgG and IgM.

23. A method of producing an antibody display library, the method comprising:
(a) introducing an antigen into a non-human somatic transgenic animal generated according to the method of claim 1;
(b) isolating a population of nucleic acids encoding one or more antibody chains from one or more lymphatic cells of that somatic transgenic animal; and
(c) forming a library from those antibody chains.

24. Hybridoma cells expressing human monoclonal antibody molecules, wherein the hybridoma cells are generated from non-human somatic-transgenic animals obtained using the method of claim 1.

25. A heterologous immunoglobulin molecule obtained by the method of claim 1.

26. A heterologous immunoglobulin molecule obtained according to claim 25 which is further characterized by at least one of the following:
a) the immunoglobulin molecule is a monoclonal antibody;
b) the immunoglobulin molecule is a fully/entirely human antibody molecule;
c) the immunoglobulin molecule is a partially human antibody molecule; and
d) the immunoglobulin molecule comprises an antibody molecule that is selected from the group consisting of: IgA, IgD, IgE, IgG and IgM.

27. A non-human somatic transgenic animal obtained using the method of claim 1, comprising nucleic acid encoding one or more heterologous immunoglobulin molecules, wherein the native/endogenous immune system of that animal is suppressed and/or functionally inactivated.

28. A non-human somatic transgenic animal comprising nucleic acid encoding one or more heterologous immunoglobulin molecules, wherein the native/endogenous immune system of that animal is suppressed and/or functionally inactivated.

29. The animal according to claim 28 wherein the heterologous immunoglobulin molecule is an antibody molecule which is at least partially human.

30. The animal according to claim 28 wherein the heterologous immunoglobulin molecule is an antibody molecule which is fully human.

31. The somatic transgenic animal according to claim 29 which is a mouse.

32. The somatic transgenic animal according to claim 31 wherein the mouse is a RAG mouse or a SCID mouse.

33. Pleuripotent cells which are committed to a B-cell fate and which comprise nucleic acid encoding one or more heterologous immunoglobulin molecules.

34. Pleuripotent cells according to claim 33 wherein the heterologous immunoglobulin molecules are antibody molecules.

35. The method of claim 1 wherein said donor cells comprise pleuripotent cells which are committed to a B-cell fate and which comprise nucleic acid encoding one or more heterologous immunoglobulin molecules.

36. A method for the treatment of a disease in a patient comprising the step of administering to that patient in need of such treatment an effective amount of a human immunoglobulin molecule obtained using the method of claim 1.

37. A method for the generation of polyclonal antibodies from a somatic transgenic animal that comprises nucleic acid encoding one or more heterologous immunoglobulin molecules, and wherein the native/endogenous immune system of that animal is suppressed and/or functionally inactivated, the method comprising immunizing said non-human somatic transgenic animal with an antigen and isolating polyclonal antisera from said animal.
